(19) United States
(12) Patent Application Publication
(10) Pub. No.: US 2024/0000947 A1
(43) Pub. Date: Jan. 4, 2024

(54) POLYPEPTIDE MODIFICATION AND CONJUGATION METHODS

(71) Applicant: Encodia, Inc., San Diego, CA (US)

(72) Inventors: Stephen VERESPY, III, San Diego, CA (US); Samantha BEASLEY, San Diego, CA (US)

(73) Assignee: Encodia, Inc., San Diego, CA (US)

(21) Appl. No.: 18/201,129

(22) Filed: May 23, 2023

Related U.S. Application Data
(60) Provisional application No. 63/345,192, filed on May 24, 2022.

(51) Int. Cl. A61K 47/54

(52) U.S. Cl. CPC A61K 47/542; A61K 47/547

(57) ABSTRACT

The present disclosure relates to a selective and efficient method to connect a polypeptide containing at least one lysine residue to a cargo moiety, where one step of the method involves reaction of a semicarbazide group with an ortho-acylphenyl boronic acid, forming a cyclic diazaborine ring fused to phenyl. Conditions for formation of the cyclic diazaborine are sufficiently mild for the method to be used in the presence of sensitive biomolecules such as polynucleotides. A substituent group on the phenyl can be used to link the cyclic diazaborine to a cargo moiety such as a polynucleotide, bead, or reactive group, providing a polypeptide—cargo moiety conjugate that is useful for various purposes, such as to analyze, identify, track, locate, detect, or immobilize the polypeptide. Also provided are polypeptide—cargo moiety conjugates, wherein the polypeptide and cargo moiety are connected via a linker that comprises a cyclic diazaborine.

### CROSS-REFERENCE TO RELATED APPLICATION

This application claims priority to U.S. Provisional Patent Application No. 63/345,192 filed May 24, 2022, entitled “POLYPEPTIDE MODIFICATION AND CONJUGATION METHODS,” which is herein incorporated by reference in its entirety for all purposes.

### TECHNICAL FIELD

The field of this invention relates to reagents and methods for producing polypeptide conjugates, such as protein-DNA conjugates, using a conjugation reaction that forms a diazaborine ring by reaction of a semicarbazide with an ortho-acylphenylboronic acid, and to conjugates formed by such methods.

### BACKGROUND

Attaching linking groups, labels, markers, and fluorogenic probes to biological molecules such as peptides and nucleic acids for the purpose of labeling the biological molecules or linking one biomolecule to another are vital to advancing our understanding of complex biological systems. Ideally, such conjugates can be formed selectively and in good yield under conditions where the biological molecules are stable and functional, e.g., in a biological medium. Methods for attaching groups to biological molecules are known, but there remains a need for new methods complementary to existing ones and methods that are more selective and efficient than those known.

Linking methods take advantage of various reactive handles that are suitable for use in complex biological systems to connect biomolecules together. These reactive handles must react under mild conditions with high selectivity in order to be useful in complex mixtures, and preferably they should function in substantially aqueous media compatible with normal structure and function of biomolecules.

A variety of methods are known for using amino acid side chains such as lysine or arginine to attach a label or cargo to the polypeptide, but most require conditions that are unduly harsh, which limits their usefulness in complex systems comprising biomolecules. Alternative conjugation strategies that operate under mild reaction conditions are needed to fully utilize amino acid side chains such as the terminal amino group of lysine as an attachment point for conjugating peptides to various cargo compounds.

### SUMMARY

The current disclosure provides a highly selective method to modify lysine residues and uses very mild conditions that are compatible with the presence of many biomolecules, including, but not limited to, nucleic acids. Moreover, the method provides a convenient way to introduce a reactive functional moiety that can be used to connect the lysine-containing polypeptide to a surface, label, or another biomolecule. The method is thus useful for making conjugates, for example, in which a lysine-containing polypeptide is attached to a polynucleotide. The method is suitable for modifying polypeptides both in solution and attached to a solid support.

In one embodiment, a method for generating conjugates from a polypeptide by attaching a functional group to a lysine residue in the polypeptide is provided. The method provides a novel method for modifying a lysine residue to convert the terminal amino group of the lysine to a semicarbazide. The semicarbazide group is then allowed to react with an ortho-acyl phenylboronic acid as shown in Scheme 1 below, where PP represents a polypeptide, LG represents a Leaving Group, and PG represents a nitrogen protecting group. Reaction of the semicarbazide with the acyl phenylboronate produces a cyclic diazaborine compound.

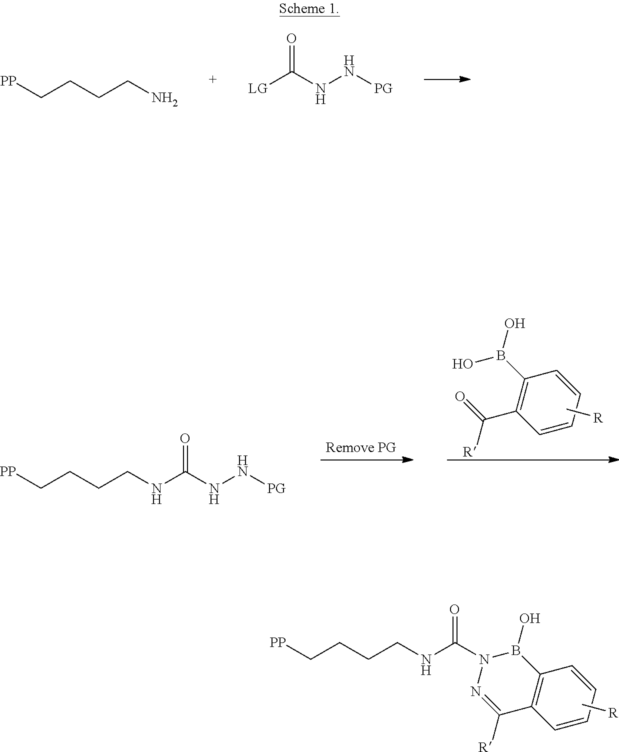

In the disclosed methods, the phenyl ring of the ortho-acyl phenylboronic acid has a substituent (represented here by R) that can include a bioorthogonal reaction handle, which can subsequently be used to attach the polypeptide to a target. The target can be a surface, a label, or another biomolecule such as a polynucleotide. The diazaborine forms efficiently and under mild conditions that are compatible with biological systems. The diazaborine forms irreversibly and is stable under conditions needed to cause the bioorthogonal handle to react with a complementary biorthogonal handle on the target, linking the polypeptide to the target. In some embodiments, the disclosed methods can be used to conjugate a lysine-containing peptide with an ortho-acyl phenyl boronic acid that is already attached to a target such as another biomolecule, bead, or surface.

In one embodiment, the present disclosure provides a method to modify a polypeptide that contains at least one lysine residue, which method comprises contacting the polypeptide with an acylating agent of Formula (I):

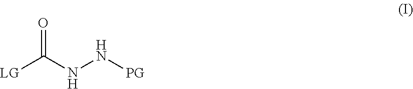

wherein:

LG is a leaving group; and PG is a nitrogen protecting group.

In preferred embodiments, the method provides a modified polypeptide comprising at least one modified lysine residue, and having the formula:

PP-(CH₂)₄—NH—C(═O)—NH—NH—PG,

wherein PP is the polypeptide;

—(CH₂)₄—NH— is the side chain of a lysine residue in the polypeptide; and PG is the nitrogen protecting group or H. In preferred embodiments, PG is the nitrogen protecting group.

In another embodiment, the present disclosure provides a conjugate comprising a polypeptide connected by a tether to a cargo moiety or a reactive functional moiety, wherein the tether comprises a diazaborine, and wherein the conjugate has the formula:

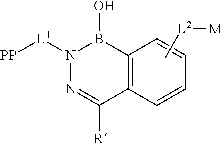

wherein:

R′ is H or C₁₋₄ alkyl;

PP is the polypeptide;

M is the cargo moiety or the reactive functional moiety;

L¹ is a linker connecting the tether to PP; and

L² is a linker connecting the tether to M.

In yet another embodiment, the present disclosure provides a method for preparing a conjugate having the formula:

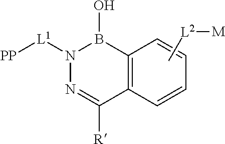

wherein: R′ is H or C₁₋₄ alkyl; PP is a polypeptide, which is connected by a tether to M, wherein the tether comprises a diazaborine; M is a cargo moiety or a reactive functional moiety that is configured to connect the conjugate to a cargo moiety; L¹ is a linker connecting the tether to PP; and L² is a linker connecting the tether to M;
the method comprises the following steps:

a. modifying a polypeptide that contains at least one lysine residue by a method which comprises contacting the polypeptide with an acylating agent of Formula (I):

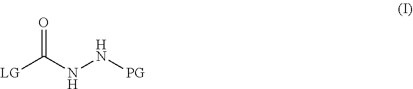

wherein:

LG is a leaving group; and PG is a nitrogen protecting group, to attach a semicarbazide group to the at least one lysine residue to form a polypeptide semicarbazide compound;

b. optionally, removing the nitrogen protecting group present on the semicarbazide group of the polypeptide semicarbazide compound; and
c. contacting the polypeptide semicarbazide compound with an ortho-acyl phenylboronic acid of the formula:

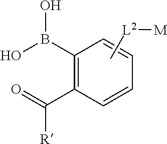

under conditions where the semicarbazide group reacts with the ortho-acyl phenylboronic acid to form a diazaborine to provide the conjugate.

In yet another embodiment, the present disclosure provides a method of analyzing a polypeptide comprising at least one lysine residue, the method comprising the steps of:

a. providing a conjugate of the polypeptide and a recording tag on a solid support, wherein the recording tag comprises a polynucleotide that is conjugated to the polypeptide according to the following steps:
(i) contacting the polypeptide with an acylating agent of Formula (I)

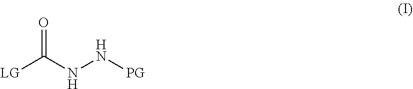

wherein: LG is a leaving group; and PG is a nitrogen protecting group, to attach a semicarbazide group to the at least one lysine residue to form a polypeptide semicarbazide compound;
(ii) optionally, removing any protecting group present on the semicarbazide group of the polypeptide semicarbazide compound; and
(iii) contacting the polypeptide semicarbazide compound with an ortho-acyl phenylboronic acid of the formula:

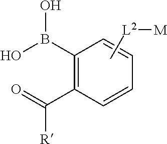

wherein L² is a linking group and M is the recording tag or a reactive functional moiety that is configured to connect the conjugate to the recording tag, under conditions where the semicarbazide group reacts with the ortho-acyl phenylboronic acid to form a diazaborine to provide the conjugate;
(iv) optionally, when M is the reactive functional moiety, using the reactive functional moiety to connect the conjugate to the recording tag;
(v) attaching the polypeptide or the conjugate to the solid support before or after any one of the steps (i)-(iv);

b. contacting the polypeptide with a binding agent capable of binding to the polypeptide, wherein the binding agent comprises (i) a coding tag that comprises identifying information regarding the binding agent; or (ii) a detectable label; and

c. analyzing the polypeptide by (i) obtaining signal from detectable label upon binding of the binding agent to the polypeptide; or (ii) c1) transferring the identifying information from the coding tag to the recording tag to generate an extended recording tag; and c2) analyzing the extended recording tag.

In preferred embodiments, analyzing the polypeptide comprises identifying at least partial amino acid sequence of the polypeptide.

The methods described herein can be used for any suitable purpose. In some embodiments, the conjugation methods are used to generate conjugates comprising a polypeptide linked to a polynucleic acid. They are suitable for use in preparing polypeptide samples for analysis and for preparing libraries of polypeptide conjugates, which are useful in methods such as those disclosed in US 20190145982 A1 for high throughput analysis of polypeptides.

### BRIEF DESCRIPTION OF THE DRAWINGS

FIG. 1 shows an exemplary workflow for modification of a lysine residue in a polypeptide using the disclosed methods, based on the following steps: 1) reacting the side chain of a lysine residue of a polypeptide with a semicarbazide transfer reagent generating a semicarbazide-modified polypeptide; 2) reacting the semicarbazide-modified polypeptide with 2-formylphenylboronic acid (FPBA) conjugated with a reactive handle to form a modified polypeptide having diazaborine moiety and the reactive handle; 3) conjugating the polypeptide with a cargo moiety (such as polynucleotide) using the reactive handle.

FIG. 2 shows exemplary synthesis of FPBA-N₃ reagent.

FIG. 3A-FIG. 3B show analysis of exemplary conjugation reactions performed under different conditions. The samples analyzed were as follows: (A)—serum stock (protein only); (B)—SC-modified serum proteins (SC=semicarbazide) conjugated with FPBA-N₃; (C)—SC-modified serum proteins conjugated with FPBA-DNA; (D)—SC-modified serum proteins treated with 1.4M Urea and Trypsin (in 0.1M Tris at pH=8), then conjugated with FPBA-DNA; (E)—SC-modified serum proteins treated with Trypsin (in 0.1M Tris at pH=8), then conjugated with FPBA-DNA; (F)—DBCO-DNA only. FIG. 3A shows analysis of the corresponding samples using gel electrophoresis (200 V, 50 min) in 16% Tris-Gly Protein Gel, whereas FIG. 3B shows analysis of the corresponding samples using gel electrophoresis (200 V, 20 min) in 15% TBU DNA Gel.

FIG. 4 shows an exemplary workflow for conjugation of semicarbazide-modified polypeptides to the FPBA-oligonucleotide fusions each attached to a solid support (bead).

FIG. 5 shows results of exemplary conjugation between semicarbazide-modified polypeptides from serum samples and the FPBA-oligonucleotide fusions attached to beads (see Example 2 for details). DNA molecules in samples were resolved in 15% TBU DNA gel (200 V, 20 min) and stained with SYBR Gold stain. Lane (1)—ssDNA ladder; Lane (2) -oligonucleotide-only sample before conjugation with the semicarbazide-labeled polypeptides; Lane (3)—oligonucleotide-polypeptides conjugates after the FPBA functionalization and SC-polypeptides conjugation (shows high molecular weight (HMW) fraction formed). Arrows indicate DNA and 2-FPBA-N3-DNA that were not conjugated to SC-peptides.

FIG. 6 shows an exemplary workflow for N-terminal peptide capture, lysine semicarbazide (SC) modification of peptides on a solid support and subsequest release under mild conditions, followed by immobilization on another solid support through formylphenylboronic acid (FPBA) conjugation (diazaborine formation) for ProteoCode™ assay. The following steps are shown: N-terminal (NT) peptide immobilization; lysine modification with SC transfer reagent; semicarbazide deprotection; SC-modified peptide release; and conjugation to FPBA-DNA hairpin immobilized on a solid support.

### DETAILED DESCRIPTION

Non-limiting embodiments of the present invention will be described below by way of examples with reference to the accompanying figures, which are intended to illustrate some variations of the methods and compositions of the invention. For purposes of illustration, not every component is labeled in every figure, nor is every component of each embodiment of the invention shown where illustration is not necessary to allow those of ordinary skill in the art to understand the Figures and the invention.

The following description and examples are intended to illustrate and exemplify certain aspects and embodiments of the invention but are not intended to limit its scope.

### Definitions

Unless defined otherwise, all technical and scientific terms used herein have the same meaning as is commonly understood by one of ordinary skill in the art to which this invention belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein, “a” or “an” means “at least one” or “one or more”.

The term “comprising” as used herein takes its conventional meaning as used for patent purposes, and is considered an open transition. It thus refers to methods or compositions that include specified features and may optionally include ones. Thus, a method ‘comprising’ steps (1) and (2) can optionally include additional steps such as (3) and (4), etc. Similarly, a composition comprising components (1) and (2) can optionally include additional components such as (3) and (4), etc. The term “consisting of” as used herein takes its conventional meaning as used for patent purposes, and is considered a closed transition. It thus refers to methods or compositions that include specified steps or features and no additional ones. The term “consisting essentially of” as used herein takes its conventional meaning as used for patent purposes, and refers to methods or compositions that include specified steps or features and may include additional steps or features that do not materially change the product or process described by the explicit steps or features.

An aspect of the invention described as ‘comprising’ certain features is intended to disclose and to include the aspects of the invention ‘consisting of’ and/or ‘consisting essentially of’ the recited features.

The term “alkyl” as used herein refers to saturated hydrocarbon groups in a straight, branched, or cyclic configuration or any combination thereof, and particularly contemplated alkyl groups include those having ten or less carbon atoms, especially 1-6 carbon atoms and lower alkyl groups having 1-4 carbon atoms. Exemplary alkyl groups are methyl, ethyl, propyl, isopropyl, butyl, sec-butyl, tertiary butyl, pentyl, isopentyl, hexyl, cyclopropylmethyl, etc.

The term “alkenyl” as used herein refers to an alkyl as defined above having at least two carbon atoms and at least one carbon-carbon double bond. Thus, particularly contemplated alkenyl groups include straight, branched, or cyclic alkenyl groups having two to ten carbon atoms (e.g., ethenyl, propenyl, butenyl, pentenyl, etc.) or 5-10 atoms for cyclic alkenyl groups. Alkenyl groups are optionally substituted by groups suitable for alkyl groups as set forth herein.

Similarly, the term “alkynyl” as used herein refers to an alkyl or alkenyl as defined above and having at least two (preferably three) carbon atoms and at least one carbon-carbon triple bond. Especially contemplated alkynyls include straight, branched, or cyclic alkynes having two to ten total carbon atoms (e.g., ethynyl, propynyl, butynyl, cyclopropylethynyl, etc.). Alkynyl groups are optionally substituted by groups suitable for alkyl groups as set forth herein.

The term “cycloalkyl” as used herein refers to a cyclic alkane (i.e., in which a chain of carbon atoms of a hydrocarbon forms a ring), preferably including three to eight carbon atoms. Thus, exemplary cycloalkanes include cyclopropyl, cyclobutyl, cyclopentyl, cyclohexyl, cycloheptyl, and cyclooctyl. Cycloalkyls also include one or two double bonds, which form the “cycloalkenyl” groups. Cycloalkyl groups are optionally substituted by groups suitable for alkyl groups as set forth herein.

Aromatic groups containing one or more heteroatoms (typically N, O or S) as ring members can be referred to as heteroaryl or heteroaromatic groups. Typical heteroaromatic groups include monocyclic C5-C6 aromatic groups such as pyridyl, pyrimidyl, pyrazinyl, thienyl, furanyl, pyrrolyl, pyrazolyl, thiazolyl, oxazolyl, isothiazolyl, isoxazolyl, and imidazolyl and the fused bicyclic moieties formed by fusing one of these monocyclic groups with a phenyl ring or with any of the heteroaromatic monocyclic groups to form a C8-C10 bicyclic group such as indolyl, benzimidazolyl, indazolyl, benzotriazolyl, isoquinolyl, quinolyl, benzothiazolyl, benzofuranyl, pyrazolopyridyl, pyrazolopyrimidyl, quinazolinyl, quinoxalinyl, cinnolinyl, and the like. Any monocyclic or fused ring bicyclic system which has the characteristics of aromaticity in terms of electron distribution throughout the ring system is included in this definition. It also includes bicyclic groups where at least the ring which is directly attached to the remainder of the molecule has the characteristics of aromaticity. Typically, the ring systems contain 5-12 ring member atoms.

As also used herein, the terms “heterocycle”, “cycloheteroalkyl”, and “heterocyclic moieties” are used interchangeably herein and refer to any compound in which a plurality of atoms form a ring via a plurality of covalent bonds, wherein the ring includes at least one atom other than a carbon atom as a ring member. Particularly contemplated heterocyclic rings include 5- and 6-membered rings with nitrogen, sulfur, or oxygen as the non-carbon atom (e.g., imidazole, pyrrole, triazole, dihydropyrimidine, indole, pyridine, thiazole, tetrazole etc.). Typically these rings contain 0-1 oxygen or sulfur atoms, at least one and typically 2-3 carbon atoms, and up to four nitrogen atoms as ring members. Further contemplated heterocycles may be fused (i.e., covalently bound with two atoms on the first heterocyclic ring) to one or two carbocyclic rings or heterocycles, and are thus termed “fused heterocycle” or “fused heterocyclic ring” or “fused heterocyclic moieties” as used herein. Where the ring is aromatic, these can be referred to herein as ‘heteroaryl’ or heteroaromatic groups.

The term “haloalkyl” refers to an alkyl group as described above, wherein one or more hydrogen atoms on the alkyl group have been substituted with a halo group. Examples of such groups include, without limitation, fluoroalkyl groups, such as fluoroethyl, trifluoromethyl, difluoromethyl, trifluoroethyl and the like.

The term “haloalkoxy” refers to the group alkyl-O- wherein one or more hydrogen atoms on the alkyl group have been substituted with a halo group and include, by way of examples, groups such as trifluoromethoxy, and the like.

The term “alkoxy” as used herein refers to a hydrocarbon group connected through an oxygen atom, e.g., —O-Hc, wherein the hydrocarbon portion Hc may have any number of carbon atoms, typically 1-10 carbon atoms, may further include a double or triple bond and may include one or two oxygen, sulfur or nitrogen atoms in the alkyl chains, and can be substituted with aryl, heteroaryl, cycloalkyl, and/or heterocyclyl groups. For example, suitable alkoxy groups include methoxy, ethoxy, propyloxy, isopropoxy, methoxyethoxy, benzyloxy, allyloxy, and the like. Similarly, the term “alkylthio” refers to alkylsulfides of the general formula —S-Hc, wherein the hydrocarbon portion Hc is as described for alkoxy groups. For example, contemplated alkylthio groups include methylthio, ethylthio, isopropylthio, methoxyethylthio, benzylthio, allylthio, and the like.

The term ‘amino’ as used herein refers to the group —NH₂. The term “alkylamino” refers to amino groups where one or both hydrogen atoms are replaced by a hydrocarbon group Hc as described above, wherein the amino nitrogen “N” can be substituted by one or two Hc groups as set forth for alkoxy groups described above. Exemplary alkylamino groups include methylamino, dimethylamino, ethylamino, diethylamino, etc. Also, the term “substituted amino” refers to amino groups where one or both hydrogen atoms are replaced by a hydrocarbon group Hc as described above, wherein the amino nitrogen “N” can be substituted by one or two Hc groups as set forth for alkoxy groups described above.

The term ‘acyl’ as used herein refers to a group of the formula —C(═O)-D, where D is an alkyl, alkenyl, alkynyl, cycloalkyl, aryl, heteroaryl, or heterocycle as described above. Typical examples are groups wherein D is a C1-C10 alkyl, C2-C10 alkenyl or alkynyl, or phenyl, each of which is optionally substituted. In some embodiments, D can be H, Me, Et, isopropyl, propyl, butyl, C1-C4 alkyl substituted with —OH, —OMe, or NH₂, phenyl, halophenyl, alkylphenyl, and the like.

The term “aryloxy” as used herein refers to an aryl group connecting to an oxygen atom, wherein the aryl group may be further substituted. For example, suitable aryloxy groups include phenyloxy or phenoxy, etc. Similarly, the term “arylthio” as used herein refers to an aryl group connecting to a sulfur atom, wherein the aryl group may be further substituted. For example, suitable arylthio groups include phenylthio, etc.

The hydrocarbon portion of each alkoxy, alkylthio, alkylamino, and aryloxy, etc. can be substituted as appropriate for the relevant hydrocarbon moiety.

It should further be recognized that all of the above-defined groups may further be substituted with one or more substituents, which may in turn be substituted with hydroxy, amino, cyano, C1-C4 alkyl, halo, or C1-C4 haloalkyl. For example, a hydrogen atom in an alkyl or aryl can be replaced by an amino, halo or C1-4 haloalkyl or alkyl group.

The term “substituted” as used herein refers to a replacement of a hydrogen atom of the unsubstituted group with a functional group, and particularly contemplated functional groups include nucleophilic groups (e.g., —NH₂, —OH, —SH, —CN, etc.), electrophilic groups (e.g., C(O)OR, C(X)OH, etc.), polar groups (e.g., —OH), non-polar groups (e.g., heterocycle, aryl, alkyl, alkenyl, alkynyl, etc.), ionic groups (e.g., —NH₃₊), and halogens (e.g., —F, —Cl), NHCOR, NHCONH₂, OCH₂COOH, OCH₂CONH₂, OCH₂CONHR, NHCH₂COOH, NHCH₂CONH₂, NHSO₂R, OCH₂-heterocycles, POSH, SO₃H, amino acids, and all chemically reasonable combinations thereof. Moreover, the term “substituted” also includes multiple degrees of substitution, and where multiple substituents are disclosed or claimed, the substituted compound can be independently substituted by one or more of the disclosed or claimed substituent moieties.

In addition to the disclosure herein, in a certain embodiment, a group that is substituted has 1, 2, 3, or 4 substituents, 1, 2, or 3 substituents, 1 or 2 substituents, or 1 substituent.

It is understood that in all substituted groups defined above, compounds arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups specifically contemplated herein are limited to substituted aryl-(substituted aryl)-substituted aryl.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

The term “salt thereof” means a compound formed when a proton of an acid is replaced by a cation, such as a metal cation or an organic cation and the like, as well as a compound formed when a basic group in a compound accepts a proton or additional group causing the compound to have a positive charge, and which is thus associated with an anionic counterion such as a halide, nitrate, sulfate, carbonate, carboxylate, and the like. Where applicable, the salt is a pharmaceutically acceptable salt, although this is not required for salts of compounds that are not intended for administration to a patient. By way of example, salts of the present compounds include those wherein the compound is protonated by an inorganic or organic acid to form a cation, with the conjugate base of the inorganic or organic acid as the anionic component of the salt, as well as compounds protonated or alkylated and the like.

As used herein, the term “bioorthogonal reactive handle” or “bioorthogonal handle” refers to a reactive moiety that is stable in typical biological media and systems, and reacts specifically with appropriate non-biological reactive groups under mild conditions that do not damage the biological system. Examples of bioorthogonal handles include tetrazines (which can participate in ‘click’ reactions with strained alkenes and alkynes such as cyclopropenes, trans-cyclooctene, cyclooctyne, and the like); alkyl azides (which take part in ‘click’ reactions with terminal alkynes and alkenes); phosphines and azides (which can take part in Staudinger ligation reactions to form amide bonds). Examples of bioorthogonal handles and strategies for using them are well known in the art. See e.g., C. P. Ramil, et al., Chem. Commun. 2013, vol. 49, 11007-11022; M. F. Debets, et al., Org. Biomol. Chem. 2013, vol. 11, 6439.

As used herein, the term “inverse diene” refers to an electrone poor diene capable of reacting with an electron-rich multiple bond in an inverse-electron demand Diels-Alder reaction, such as a 1,2,4,5-tetrazine.

As used herein, the term “Diazaborine” refers to a cyclic group containing a “B-N-N” linkage, e.g.

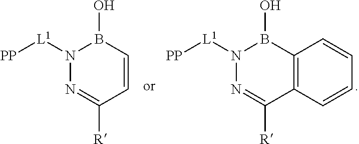

Note that these compounds can be further substituted, and can be depicted in alternative resonance forms such as

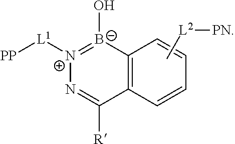

The alternative resonance forms are included in the depicted “uncharged” forms of diazaborine rings.

The term “conjugation reagent” as used herein refers to an organic moiety that can be used to, or is used to, connect (link) two moieties. It typically includes at least one reactive handle useful for attaching to a target moiety. Examples include connecting a chosen moiety to at least one other molecular component, such as a reactive handle, functional group, label, binding group, tag, or target compound. A conjugation reagent can be substituted with various groups such as reactive handles and/or detectable labels. For example, a first target compound such as a lysine-containing peptide can be covalently attached to a conjugation reagent via the primary amine of a lysine group; in the disclosed methods, the lysine can be reacted with a reagent to convert it to a semicarbazide to form a ‘first moiety—conjugation reagent’ conjugate. With a conjugation reagent that contains an additional reactive handle that remains intact when the conjugation reagent reacts with a first target molecule, the additional reactive handle can be used to connect the ‘first target—conjugation reagent’ conjugate to a second target compound that contains a functional group that is suitable to react with the additional reactive handle of the conjugation reagent. An example of this is use of a substituted ortho-acyl phenylboronic acid such as those described herein, when the substituent on the phenyl ring has a biorthogonal reactive handle. For simplicity, the resulting product can be described as a ‘first target—conjugation reagent—second target’ conjugate, even though the conjugation reagent is modified by the reactions it participated in to form the conjugate. The person of ordinary skill will understand that some of the reactive group structures change during the course of reactions that occur as part of the methods described herein, and while a conjugation reagent attached to a target compound has a structure that is necessarily modified during attachment it is still referred to as a conjugation reagent or linking group.

The term “target compound” or “target moiety” as used herein refers to a compound that is to be used in the methods herein to form a conjugate, and particularly to be covalently attached to a linker or conjugation reagent. Typical target compounds include peptides, nucleic acids, oligosaccharides, lipopolysaccharides, and other macromolecules such as combinations of one or more of these, as well as polymers and small-molecules (up to about MW 1500).

Unless otherwise described, a conjugation reagent can comprise one or more groups selected from a nucleotide, a nucleotide analog, an amino acid, a peptide, a polypeptide, aryl ring, heteroaryl ring, heterocyclic ring, carbocyclic ring, one or more polyethylene glycol (PEG) subunits including a PEG chain containing up to 100 or more PEG units. A conjugation reagent may be used to join a binding agent with a coding tag, a recording tag with a macromolecule (e.g., peptide), a macromolecule with a solid support, a recording tag with a solid support, etc. In certain embodiments, a conjugation reagent joins two molecules via enzymatic reaction or chemical reaction (e.g., click chemistry).

The term “conjugate” as used herein refers to a conjugate wherein one type of macromolecule is tethered to another type of macromolecule. A class of conjugates of particular interest involves a polypeptide tethered to a polynucleotide.

Conjugation reagents that comprise a detectable label are sometimes referred to herein as “probes” or “fluorogenic probes.”

The term “modifier” as used herein refers to a chemical moiety that can usefully be chemically attached to a target compound to modify the structure and properties of the target compound; the modifier comprises at least one reactive handle. An example used in some embodiments of the disclosed methods is a reagent to convert a free amine of a lysine residue into a semicarbazide: these reagents enable the modified polypeptide to subsequently react with an ortho-acylphenyl boronic acid as shown herein.

The term “acylated NH₂” as used herein refers to an NH₂ group that is attached to a C=X group, where X is O, S or NR, where R is H or C₁₋₄ alkyl. Acylated NH₂ groups include guanidine, urea, thiourea, and amidine groups.

The term “detectable label” as used herein refers to a substance which can indicate the presence of another substance when associated with it. The detectable label can be a substance that is linked to or incorporated into the substance to be detected. In some embodiments, a detectable label is suitable for allowing for detection and also quantification, for example, a detectable label that emitting a detectable and measurable signal. Examples of detectable labels include a dye, a fluorophore, a chromophore, a fluorescent nanoparticle (e.g. quantum dot), a radiolabel, an enzyme (e.g. alkaline phosphatase, luciferase or horseradish peroxidase), or a chemiluminescent or bioluminescent molecule.

The term “aqueous medium” as used herein refers to a solvent or solvent mixture that is predominantly water, e.g., at least 50% water by volume. The aqueous medium can include one or more co-solvents, including organic co-solvents such as acetonitrile, DMSO, DMF, DMA, NMP, TMU, cyrene, sulfolane, 2-methyl THF, limonene, 1,3-dimethylpyridone, THF, dioxane, DME, alcohols such as methanol, ethanol, isopropanol, t-butanol, n-butanol, ethylene glycol, propylene glycol, polyethylene glycol, and the like. In some embodiments, the aqueous medium comprises 1-25% organic cosolvent such as those just named, or a mixture of those. In some embodiments, the aqueous medium comprises 1-10% organic cosolvents. In some embodiments, the aqueous medium comprises 10-20% organic cosolvents.

As used herein, the term “reactive handle” or “reactive functional moiety” refers to a moiety on a first molecule that can be caused to react with a second molecule having a complementary ‘reactive handle’ to form a covalent bond between the first molecule and the second molecule. The first and second reactive handles thus combine to form part of a tether connecting the first and second molecules. Typical reactive handles include functional groups such as carboxylate groups and amines, which can react with each other to form amides; thiols and alkylating reagents that can be reacted to form thioethers; thiols and maleimides that can be reacted to form thiosuccinimides; strained alkenes or alkynes and 1,3-dipoles such as azides that can react via cycloaddition reactions, e.g., copper-free click chemistry; and tetrazines that can react via inverse-electron demand Diels-Alder chemistry with electron rich or strained alkenes and alkynes.

For each reactive handle, there is a complementary reactive handle that will react with it to form a covalent linkage. A ‘complementary reactive handle’ as used herein refers to one of a pair of reactive handles that react with each other. Many examples are known, see e.g., M. F. Debets, et al., Org. Biomol. Chem. 2013, vol. 11, 6439. For example, an alkyl azide is a complementary reactive handle that can be used with a terminal alkyne: the alkyl azide and terminal alkyne can react to form a triazole ring, and the reaction can be used to connect two compounds together. Tetrazines are well known reactive handles: they can react in ‘tetrazine ligation’ reactions with a variety of complementary reactive handles, e.g., norbornenes, cyclooctynes, and trans-cyclooctenes:

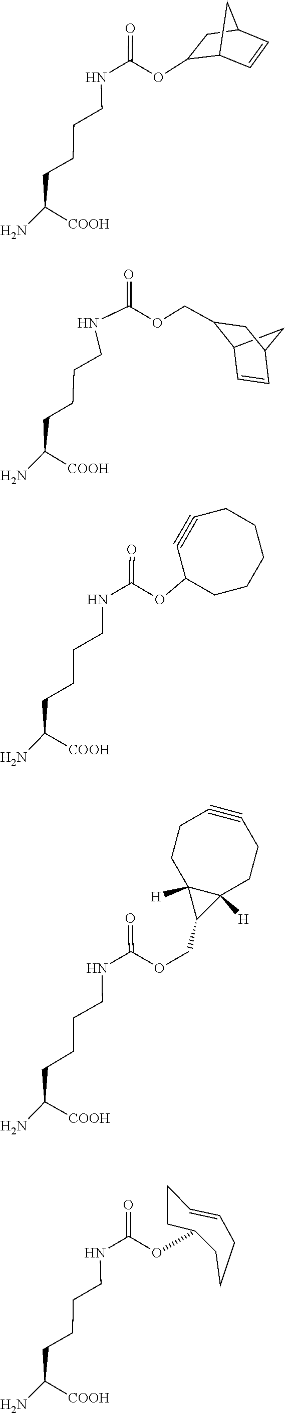

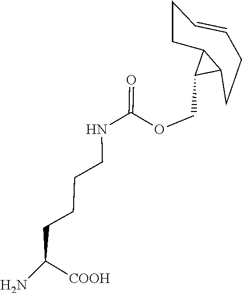

C. P. Ramil, et al., Chem. Commun. 2013, vol. 49, 11007-11022.

“Bioorthogonal” reactive handles are reactive handles that can be used in biological systems, i.e., in aqueous media, and that are generally not reactive toward common functional groups in the biological system, so they can be used to manipulate biological compounds selectively, without interference from the biomolecule components. Bioorthogonal chemistry is well known in the art: suitable functional groups for bioorthogonal chemistry include ketones, aldehydes, hydrazides, alkoxyamines, azides, terminal alkynes, phosphines, nitrones, nitrile oxides, diazo compounds, tetrazines, tetrazoles, quadrocyclanes, alkenes, iodobenzenes, transcyclooctenes, cyclooctynes, norbornenes, cyclopropenes, vinyls, isonitriles, and cycloaddition reactants. M. F. Debets, et al., Org. Biomol. Chem. 2013, vol. 11, 6439. Examples include click chemistry, particularly copper-free click chemistry, which uses cycloaddition reactants like cyclooctyne that react efficiently with alkyl azides; and inverse-electron demand Diels-Alder chemistries such as tetrazines, which react with strained alkenes or alkynes like cyclopropene and trans-cyclooctene as well as strained alkynes like cyclooctynes. Useful cyclooctynes include:

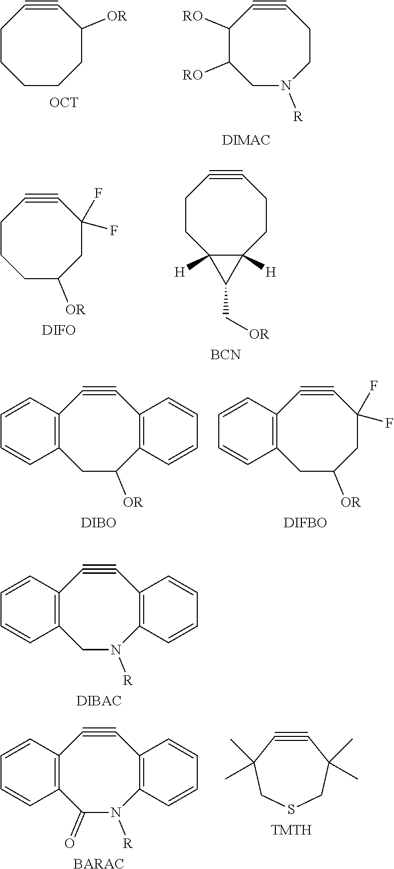

‘R’ in these structures indicates where the cyclooctyne compound can be attached to a target molecule or conjugation reagent, etc. TMTH is actually a 7-membered ring, but the C—S bonds are longer than C—C bonds, so the ring strain is similar to that of a cyclooctyne. C. P. Ramil, et al., Chem. Commun. 2013, vol. 49, 11007-11022.

As used herein, the term ‘leaving group’ refers to a moiety that is readily displaced by reaction with a complementary reactant, which is often a nucleophile. In some examples herein, the leaving group is on an acyl carbon, e.g., R-C(=O)-LG, where LG is a displaceable leaving group; such acyl groups can react with a nucleophile, where the leaving group is replaced by the nucleophile. Examples of leaving groups for such acyl groups include, but are not limited to, halo, CN, azide, acyl groups such as pivaloate, alkoxyacyloxy groups such as isobutoxy-carbonyl-O, imidazole, triazole, anhydride, sulfonyl, hydrazide, sulfonylhydrazide, azobenzotriazole, pentafluorophenol, dinitrophenol, —O-benzotriazole, ethyl cyanohydroxyiminoacetate, activated alkoxy groups such as trifluorethoxy and trichloroethoxy, and —OC(O)OR where R is a C₁₋₈ alkyl. In Formula (I), some preferred leaving groups include nitrophenoxy, dinitrophenoxy, and similar phenoxy groups with electron-withdrawing groups on the phenyl ring of the phenoxy.

As used herein, the term “macromolecule” encompasses large molecules composed of smaller subunits. Examples of macromolecules include, but are not limited to peptides, polypeptides, proteins, nucleic acids, carbohydrates, lipids, macrocycles. A macromolecule also includes a chimeric macromolecule composed of a combination of two or more types of macromolecules, covalently linked together (e.g., a peptide linked to a nucleic acid). A macromolecule may also include a “macromolecule assembly”, which is composed of non-covalent complexes of two or more macromolecules. A macromolecule assembly may be composed of the same type of macromolecule (e.g., protein-protein) or of two more different types of macromolecules (e.g., protein-DNA).

As used herein, the term “peptide” is used herein interchangeably with the term “polypeptide” and refers to a molecule comprising a chain of three or more amino acids joined by peptide bonds. In general terms, a peptide having more than 20-30 amino acids is commonly referred to as a polypeptide, and one having more than 50 amino acids is commonly referred to as a protein. The amino acids of the peptide are most typically L-amino acids, but may also be D-amino acids, modified amino acids, amino acid analogs, amino acid mimetics, or any combination thereof. Peptides may be naturally occurring, synthetically produced, or recombinantly expressed. Peptides may also comprise additional groups modifying the amino acid chain, for example, functional groups added via post-translational modification.

As used herein, the term “amino acid” refers to an organic compound comprising an amine group, a carboxylic acid group, and a side-chain specific to each amino acid, which serve as a monomeric subunit of a peptide. An amino acid includes the 20 standard, naturally occurring or canonical amino acids as well as non-standard amino acids. The standard, naturally-occurring amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). An amino acid may be an L-amino acid or a D-amino acid. Non-standard amino acids may be modified amino acids, amino acid analogs, amino acid mimetics, non-standard proteinogenic amino acids, or non-proteinogenic amino acids that occur naturally or are chemically synthesized. Examples of non-standard amino acids include, but are not limited to, selenocysteine, pyrrolysine, and N-formylmethionine, (3-amino acids, Homo-amino acids, Proline and Pyruvic acid derivatives, 3-substituted alanine derivatives, glycine derivatives, ring-substituted phenylalanine and tyrosine derivatives, linear core amino acids, N-methyl amino acids.

As used herein, the term “solid support”, “solid surface”, or “solid substrate” or “substrate” refers to any solid material, including porous and non-porous materials, to which a macromolecule (e.g., peptide) can be associated directly or indirectly, by any means known in the art, including covalent and non-covalent interactions, or any combination thereof. A solid support may be two-dimensional (e.g., planar surface) or three-dimensional (e.g., gel matrix or bead). A solid support can be any support surface including, but not limited to, a bead, a microbead, an array, a glass surface, a silicon surface, a plastic surface, a filter, a membrane, nylon, a silicon wafer chip, a flow through chip, a flow cell, a biochip including signal transducing electronics, a channel, a microtiter well, an ELISA plate, a spinning interferometry disc, a polymer matrix, a nanoparticle, or a microsphere. Materials for a solid support include but are not limited to acrylamide, agarose, cellulose, nitrocellulose, glass, gold, quartz, polystyrene, polyethylene vinyl acetate, polypropylene, polymethacrylate, polyethylene, polyethylene oxide, polysilicates, polycarbonates, Teflon, fluorocarbons, nylon, silicon rubber, polyanhydrides, polyglycolic acid, polyactic acid, polyorthoesters, functionalized silane, polypropylfumerate, collagen, glycosaminoglycans, polyamino acids, dextran, or any combination thereof. For example, when solid surface is a bead, the bead can include, but is not limited to, a a ceramic bead, polystyrene bead, a polymer bead, a methylstyrene bead, an agarose bead, an acrylamide bead, a solid core bead, a porous bead, a paramagnetic bead, a glass bead, or a controlled pore bead. A bead may be spherical or an irregularly shaped. A bead's size may range from nanometers, e.g., 100 nm, to millimeters, e.g., 1 mm In certain embodiments, beads range in size from about 0.2 micron to about 200 microns, or from about 0.5 micron to about 5 micron. n some embodiments, beads can be about 1, 1.5, 2, 2.5, 2.8, 3, 3.5, 4, 4.5, 5, 5.5, 6, 6.5, 7, 7.5, 8, 8.5, 9, 9.5, 10, 10.5, 15, or 20 μm in diameter. In preferred embodiments, solid support is a bead, microparticle or microsphere having size from about 0.2 micron to about 200 microns. In certain embodiments, “a bead” solid support may refer to an individual bead or a plurality of beads.

As used herein, the term “nucleic acid molecule” or “polynucleotide” refers to a single- or double-stranded polynucleotide containing deoxyribonucleotides or ribonucleotides that are linked by 3′-5′ phosphodiester bonds, as well as polynucleotide analogs. A nucleic acid molecule includes, but is not limited to, DNA, RNA, and cDNA. A polynucleotide analog may possess a backbone other than a standard phosphodiester linkage found in natural polynucleotides and, optionally, a modified sugar moiety or moieties other than ribose or deoxyribose. Polynucleotide analogs contain bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide. Examples of polynucleotide analogs include, but are not limited to xeno nucleic acid (XNA), bridged nucleic acid (BNA), glycol nucleic acid (GNA), peptide nucleic acids (PNAs), yPNAs, morpholino polynucleotides, locked nucleic acids (LNAs), threose nucleic acid (TNA), 2′-O-Methyl polynucleotides, 2′-O-alkyl ribosyl substituted polynucleotides, phosphorothioate polynucleotides, and boronophosphate polynucleotides. A polynucleotide analog may possess purine or pyrimidine analogs, including for example, 7-deaza purine analogs, 8-halopurine analogs, 5-halopyrimidine analogs, or universal base analogs that can pair with any base, including hypoxanthine, nitroazoles, isocarbostyril analogues, azole carboxamides, and aromatic triazole analogues, or base analogs with additional functionality, such as a biotin moiety for affinity binding.

The term “sequence identity” is a measure of identity between peptides at the amino acid level, and a measure of identity between nucleic acids at nucleotide level. The peptide sequence identity may be determined by comparing the amino acid sequence in a given position in each sequence when the sequences are aligned. Similarly, the nucleic acid sequence identity may be determined by comparing the nucleotide sequence in a given position in each sequence when the sequences are aligned. “Sequence identity” means the percentage of identical subunits at corresponding positions in two sequences when the two sequences are aligned to maximize subunit matching, i.e., taking into account gaps and insertions. For example, the BLAST algorithm (NCBI) calculates percent sequence identity and performs a statistical analysis of the similarity and identity between the two sequences. The software for performing BLAST analysis is publicly available through the National Center for Biotechnology Information (NCBI) website.

The terms “corresponding to position(s)” or “position(s) . . . with reference to position(s)” of or within a peptide or a polynucleotide, such as recitation that nucleotides or amino acid positions “correspond to” nucleotides or amino acid positions of a disclosed sequence, such sequence set forth in the Sequence Listing, refers to nucleotides or amino acid positions identified in the polynucleotide or in the peptide upon alignment with the disclosed sequence using a standard alignment algorithm, such as the BLAST algorithm (NCBI). One skilled in the art can identify any given amino acid residue in a given peptide at a position corresponding to a particular position of a reference sequence, such as set forth in the Sequence Listing, by performing alignment of the peptide sequence with the reference sequence (for example, by using BLASTP publicly available through the NCBI website), matching the corresponding position of the reference sequence with the position in peptide sequence and thus identifying the amino acid residue within the peptide.

The term “peptide bond” as used herein refers to a chemical bond formed between two molecules (such as two amino acids) when the carboxyl group of one molecule reacts with the amino group of the other molecule, releasing a water molecule (H₂O).

The term “unmodified” (also “wild-type” or “native”) as used herein is used in connection with biological materials such as nucleic acid molecules and proteins, refers to those which are found in nature and not modified by human intervention.

The term “modified” or “engineered” (or “variant”, or “mutant”) as used in reference to nucleic acid molecules and protein molecules, e.g., an engineered binder or engineered cleavase enzyme, implies that such molecules are created by human intervention and/or they are non-naturally occurring. The engineered binder or engineered cleavase is a polypeptide having an altered amino acid sequence, relative to an unmodified or wild-type protein, such as starting scaffold, or a portion thereof. An engineered enzyme is a polypeptide which differs from a wild-type enzyme scaffold sequence, or a portion thereof, by one or more amino acid substitutions, deletions, additions, or combinations thereof. An engineered binder generally exhibits at least 70%, 80%, 85%, 86%, 87%, 88%, 89%, 90%, 91%, 92%, 93%, 94%, 95%, 96%, 97%, 98%, 99% or more sequence identity to a corresponding wild-type starting protein scaffold. Non-naturally occurring amino acids as well as naturally occurring amino acids are included within the scope of permissible substitutions or additions.

In some embodiments, variants of an engineered binder or engineered cleavase displaying only non-substantial or negligible differences in structure can be generated by making conservative amino acid substitutions in the engineered binder or engineered cleavase. By doing this, further engineered binder variants that comprise a sequence having at least 90% (90%, 91%, 92%, 93%, 94%, 95%, 96%, 97%, 98%, and 99%) sequence identity with the initial engineered binder sequences can be generated, retaining at least one functional activity of the engineered binder, e.g. ability to specifically bind to the N-terminally modified target peptide. Examples of conservative amino acid changes are known in the art. Examples of non-conservative amino acid changes that are likely to cause major changes in protein structure are those that cause substitution of (a) a hydrophilic residue, e.g., serine or threonine, for (or by) a hydrophobic residue, e.g., leucine, isoleucine, phenylalanine, valine or alanine; (b) a cysteine or proline for (or by) any other residue; (c) a residue having an electropositive side chain, e.g., lysine, arginine, or histidine, for (or by) an electronegative residue, e.g., glutamic acid or aspartic acid; or (d) a residue having a bulky side chain, e.g., phenylalanine, for (or by) one not having a side chain, e.g., glycine. Methods of making targeted amino acid substitutions, deletions, truncations, and insertions are generally known in the art. For example, amino acid sequence variants can be prepared by mutations in the DNA. Methods for polynucleotide alterations are well known in the art, for example, Kunkel et al. (1987) Methods in Enzymol. 154:367-382; U.S. Pat. No. 4,873,192 and the references cited therein.

The terms “specifically binding” and “specifically recognizing” are used interchangeably herein and generally refer to an engineered binder that binds to a cognate target peptide or a portion thereof more readily than it would bind to a random, non-cognate peptide. The term “specificity” is used herein to qualify the relative affinity by which an engineered binder binds to a cognate target peptide. Specific binding typically means that an engineered binder binds to a cognate target peptide at least twice more likely that to a random, non-cognate peptide (a 2:1 ratio of specific to non-specific binding). Non-specific binding refers to background binding, and is the amount of signal that is produced in a binding assay between an engineered binder and an N-terminally modified target peptide when the modified NTAA residue cognate for the engineered binder is not present at the N-terminus of the target peptide.

In some embodiments, specific binding refers to binding between an engineered binder and an N-terminally modified target peptide with a dissociation constant (Kd) of 200 nM or less.

In some embodiments, binding specificity between an engineered binder and an N-terminally modified target peptide is predominantly or substantially determined by interaction between the engineered binder and the modified NTAA residue of the N-terminally modified target peptide, which means that there is only minimal or no interaction between the engineered binder and the penultimate terminal amino acid residue (P2) of the target peptide, as well as other residues of the target peptide. In some embodiments, the engineered binder binds with at least 5 fold higher binding affinity to the modified NTAA residue of the target peptide than to any other region of the target peptide. In some embodiments, the engineered binder has a substrate binding pocket with certain size and/or geometry matching the size and/or geometry of the modified NTAA residue of the N-terminally modified target peptide, to which the engineered binder specifically binds to. In such embodiments, the modified NTAA residue occupies a volume encompassing a substrate binding pocket of the engineered binder that effectively precludes the P2 residue of the target peptide from entering into the substrate binding pocket or interacting with affinity-determining residues of the engineered binder. In some embodiments, the engineered binder specifically binds to N-terminally modified target peptides, wherein the target peptides share the same modified NTAA residue that interacts with the engineered binder, but have different P2 residues. In some embodiments, the engineered binder is capable of specifically binding to each N-terminally modified target peptide from a plurality of N-terminally modified target peptides, wherein the plurality of N-terminally modified target peptides contains at least 3, at least 5, or at least 10 N-terminally modified target peptides that were modified with the same N-terminal modifier agent, have the same modified NTAA residue, and have different P2 residues. Thus, in preferred embodiments, the engineered binder possesses binding affinity towards the modified NTAA residue of the N-terminally modified target peptide, but has little or no affinity towards P2 or other residues of the target peptide.

As used herein, “nucleic acid sequencing” means the determination of the order of nucleotides in a nucleic acid molecule or a sample of nucleic acid molecules. Similarly, “peptide sequencing” means the determination of the identity and order of at least a portion of amino acids in the peptide molecule or in a sample of peptide molecules.

As used herein, “next generation sequencing” refers to high-throughput sequencing methods that allow the sequencing of millions to billions of molecules in parallel. Examples of next generation sequencing methods include sequencing by synthesis, sequencing by ligation, sequencing by hybridization, polony sequencing, ion semiconductor sequencing, and pyrosequencing. By attaching primers to a solid substrate and a complementary sequence to a nucleic acid molecule, a nucleic acid molecule can be hybridized to the solid substrate via the primer and then multiple copies can be generated in a discrete area on the solid substrate by using polymerase to amplify (these groupings are sometimes referred to as polymerase colonies or polonies). Consequently, during the sequencing process, a nucleotide at a particular position can be sequenced multiple times (e.g., hundreds or thousands of times)—this depth of coverage is referred to as “deep sequencing.” Examples of high throughput nucleic acid sequencing technology include platforms provided by Illumina, BGI, Qiagen, Thermo-Fisher, and Roche.

As used herein, “analyzing” the peptide means to quantify, characterize, distinguish, or a combination thereof, all or a portion of the components of the peptide (e.g., partial identification of one or more amino acid residues (contiguous or non-continuous) of the peptide). For example, partial identification of amino acid residues in the peptide sequence can identify an amino acid in the protein as belonging to a subset of possible amino acids. Analysis typically begins with analysis of the n NTAA, and then proceeds to the next amino acid of the peptide (i.e., n−1, n−2, n−3, and so forth). This is accomplished by cleavage of the n NTAA, thereby converting the n−1 amino acid of the peptide to an N-terminal amino acid (referred to herein as the “n−1 NTAA”). Analyzing the peptide may also include determining the presence and frequency of post-translational modifications on the peptide, which may or may not include information regarding the sequential order of the post-translational modifications on the peptide. Analyzing the peptide may also include determining the presence and frequency of epitopes in the peptide, which may or may not include information regarding the sequential order or location of the epitopes within the peptide. Analyzing the peptide may include combining different types of analysis, for example obtaining epitope information, amino acid sequence information, post-translational modification information, or any combination thereof.

The terminal amino acid at one end of the peptide chain that has a free alpha-amino group is referred to herein as the “N-terminal amino acid” (NTAA). The terminal amino acid at the other end of the chain that has a free carboxyl group is referred to herein as the “C-terminal amino acid” (CTAA). The amino acids making up a peptide may be numbered in order, with the peptide being “n” amino acids in length. As used herein, NTAA is considered the n^{th} amino acid (also referred to herein as the “n NTAA”). Using this nomenclature, the next amino acid is the n−1 amino acid, then the n−2 amino acid, and so on down the length of the peptide from the N-terminal end to C-terminal end. In certain embodiments, an NTAA, CTAA, or both may be modified or labeled with a chemical moiety. In some embodiments, provided herein is a polypeptide that contains at least one lysine residue, wherein the lysine can be an N-terminal amino acid residue, a C-terminal amino acid residue, or an internal amino acid residue including any one or more amio acid residues in between the N-terminal amio acid residue and the C-terminal amio acid residue. In some embodiments, the polypeptide comprises an N-terminal lysine residue. In some embodiments, the polypeptide comprises a C-terminal lysine residue. In some embodiments, the polypeptide comprises one, two, three or more internal lysine residues.

As used herein, the term “coding tag” refers to a nucleic acid molecule of about 2 bases to about 100 bases, including any integer including 2 and 100 and in between, that comprises identifying information for its associated binding agent. As used herein, the term “recording tag” refers to a nucleic acid molecule of about 2 bases to about 100 bases that optionally comprises identifying information for a peptide to which it is associated. In certain embodiments, after a binding agent binds a peptide, information from a coding tag linked to a binding agent can be transferred to the recording tag associated with the peptide while the binding agent is bound to the peptide.

The compounds and substructures described herein include stable tautomers of the depicted structure as well as the structure depicted.

The term “protecting group” as used herein refers to a moiety that can be attached to a relatively reactive heteroatom such as a free amine, hydroxyl, or thiol, to prevent it from participating in certain types of reactions. Suitable protecting groups are readily attached to the heteroatom of a molecule that needs to be protected, stable under reaction conditions useful to manipulate other parts of the molecule of interest, and readily removed under appropriate conditions that do not otherwise damage the molecule being protected. Protecting groups for use on nitrogen are well known, and include carbamates, and certain acyl and sulfonyl groups that form amides and sulfonamides. Particularly useful nitrogen protecting groups for use in the discosed methods and in compounds of Formula (I) include tert-butoxy carbamate, fluorenylmethoxy carbamate, a dimethylaminoxy carbamate, 2-(trimethylsilyl)ethoxycarbamate, 2-(trimethylsilyl)ethanesulfonamide, and picoloyl amides. In some embodiments, exemplary protecting groups for use on a nitrogen includes, but are not limited to, ester that forms a carbamate with the nitrogen, acyl that forms an amide with the nitrogen, and sulfonyl that forms a sulfonamide with the nitrogen. In preferred embodiments, the protecting group (PG) itself does not include the nitrogen.

The present invention uses reagents and methods that selectively react with and modify a lysine residue in a protein, such as lysine residues located on a surface of the protein molecule (solvent-exposed lysine residues). The invention provides reactions useful to modify a protein that comprises at least one lysine residue, by transforming the terminal amino group of the lysine side chain to form a semicarbazide, and reaction conditions to perform this modification under conditions mild enough to preserve integrity of biomolecules, such as polynucleotides or polypeptides having post-translational modifications. It further provides methods to attach a reactive functional moiety or cargo molecule to a functionalized protein comprising the modified lysine residue, as well as compositions that comprise the functionalized protein and protein conjugates having a linker that comprises a diazaborine moiety.

In one embodiment, the present disclosure provides a method to modify a polypeptide that contains at least one lysine residue, which method comprises contacting the polypeptide with an acylating agent of Formula (I):

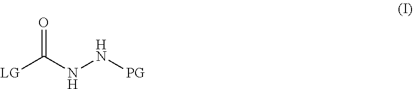

wherein:

LG is a leaving group; and PG is a nitrogen protecting group.

In preferred embodiments, the method provides a modified polypeptide comprising at least one modified lysine residue, and having the formula:

PP—(CH₂)₄—NH—C(=O)—NH—NH—PG,

wherein PP is the polypeptide;

—(CH₂)₄—NH— is the side chain of a lysine residue in the polypeptide; and PG is the nitrogen protecting group.

In some embodiments, the reacting step is performed in a buffer having a pH about 7.5 to about 11.5.

In another embodiment, the present disclosure provides a method for C-terminal N-functionalization of a polypeptide, comprising reacting the peptide of Formula:

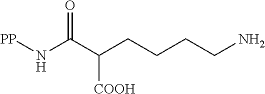

or a salt thereof, wherein PP is a polypeptide, with an acylating agent of Formula (I):

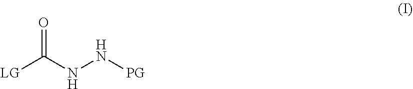

wherein LG is a leaving group, and PG is a nitrogen protecting group, in a buffer having a pH about 7.5 to about 11.5, to obtain a modified polypeptide comprising at least one modified lysine residue, and having the formula:

PP—(CH₂)₄—NH—C(=O)—NH—NH—PG,

wherein PP is the polypeptide;

—(CH₂)₄—NH— is the side chain of a lysine residue in the polypeptide; and PG is the nitrogen protecting group.

In yet another embodiment, the present disclosure provides a conjugate comprising a polypeptide connected by a tether to a cargo moiety or a reactive functional moiety, wherein the tether comprises a diazaborine, and wherein the conjugate has the formula:

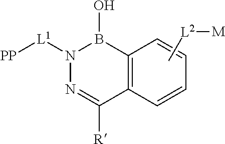

wherein:

R′ is H or C₁₋₄ alkyl;

PP is the polypeptide;

M is the cargo moiety or the reactive functional moiety;

L¹ is a linker connecting the tether to PP; and

L² is a linker connecting the tether to M.

In some embodiments of the conjugate, -L²-M is of Formula —O—Z—CC, wherein Z is C₂-C₁₂ alkylene, and CC is a bioorthogonal handle. In some embodiments, CC comprises a click chemistry reactant.

In yet another embodiment, the present disclosure provides a method for preparing a conjugate having the formula:

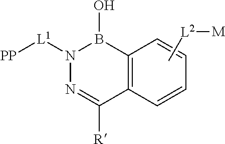

wherein: R′ is H or C₁₋₄ alkyl; PP is a polypeptide, which is connected by a tether to M, wherein the tether comprises a diazaborine; M is a cargo moiety or a reactive functional moiety that is configured to connect the conjugate to a cargo moiety; L¹ is a linker connecting the tether to PP; and L² is a linker connecting the tether to M;
the method comprises the following steps:

a. modifying a polypeptide that contains at least one lysine residue by a method which comprises contacting the polypeptide with an acylating agent of Formula (I):

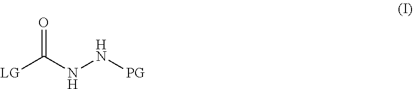

wherein:

LG is a leaving group; and PG is a nitrogen protecting group, to attach a semicarbazide group to the at least one lysine residue to form a polypeptide semicarbazide compound;

b. optionally, removing the nitrogen protecting group present on the semicarbazide group of the polypeptide semicarbazide compound; and

c. contacting the polypeptide semicarbazide compound with an ortho-acyl phenylboronic acid of the formula:

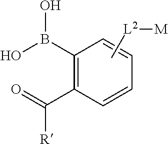

under conditions where the semicarbazide group reacts with the ortho-acyl phenylboronic acid to form a diazaborine to provide the conjugate.

In yet another embodiment, the present disclosure provides a conjugate having a polypeptide connected via a lysine residue of the polypeptide to a diazaborine, wherein the conjugate has the formula:

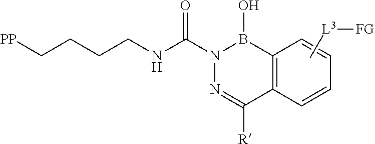

wherein:

R′ is H or C₁₋₄ alkyl;

PP is the polypeptide;

FG is a reactive functional moiety suitable for attaching the conjugate to a cargo moiety; and L³ is a linker connecting the diazaborine to FG.

In yet another embodiment, the present disclosure provides a method of analyzing a polypeptide comprising at least one lysine residue, the method comprising the steps of:

(a) providing a conjugate of the polypeptide and a recording tag on a solid support, wherein the recording tag comprises a polynucleotide that is conjugated to the polypeptide according to the following steps:

(i) contacting the polypeptide with an acylating agent of Formula (I)

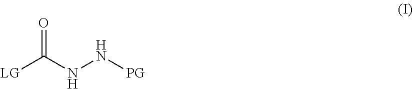

wherein: LG is a leaving group; and PG is a nitrogen protecting group, to attach a semicarbazide group to the at least one lysine residue to form a polypeptide semicarbazide compound;

(ii) optionally, removing any protecting group present on the semicarbazide group of the polypeptide semicarbazide compound; and

(iii) contacting the polypeptide semicarbazide compound with an ortho-acyl phenylboronic acid of the formula:

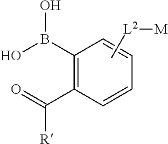

wherein L² is a linking group and M is the recording tag or a reactive functional moiety that is configured to connect the conjugate to the recording tag, under conditions where the semicarbazide group reacts with the ortho-acyl phenylboronic acid to form a diazaborine to provide the conjugate;

(iv) optionally, when M is the reactive functional moiety, using the reactive functional moiety to connect the conjugate to the recording tag;

(v) attaching the polypeptide or the conjugate to the solid support before or after any one of the steps (i)-(iv);

(b) contacting the polypeptide with a binding agent capable of binding to the polypeptide, wherein the binding agent comprises (i) a coding tag that comprises identifying information regarding the binding agent; or (ii) a detectable label; and
(c) analyzing the polypeptide by (i) obtaining signal from detectable label upon binding of the binding agent to the polypeptide; or (ii) c1) transferring the identifying information from the coding tag to the recording tag to generate an extended recording tag; and c2) analyzing the extended recording tag

In yet another embodiment, the present disclosure provides a method of analyzing a polypeptide comprising at least one lysine residue, the method comprising the steps of:

a. providing a conjugate of the polypeptide and a recording tag, the conjugate attached to a solid support, wherein the recording tag comprises a polynucleotide that is conjugated to the polypeptide according to the following steps:
(i) contacting the polypeptide with an acylating agent of Formula (I)

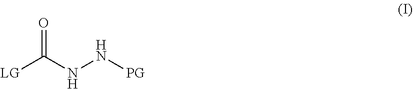

to attach a semicarbazide group to the at least one lysine residue to form a polypeptide semicarbazide compound, e.g., by a method according to any one of the embodiments provided herein;
(ii) optionally, removing any protecting group present on the semicarbazide group of the polypeptide semicarbazide compound; and
(iii) contacting the polypeptide semicarbazide compound with an ortho-acyl phenylboronic acid of the formula:

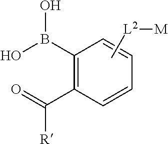

wherein L² is a linking group and M is the recording tag or a reactive functional moiety that is configured to connect the conjugate to the recording tag, under conditions where the semicarbazide group reacts with the ortho-acyl phenylboronic acid to form a diazaborine to provide the conjugate;
(iv) optionally, when M is the reactive functional moiety, using the reactive functional moiety to connect the conjugate to the recording tag;
(v) attaching the polypeptide or the conjugate to the solid support before or after any one of the steps (i)-(iv);

b. contacting the polypeptide of the conjugate with a binding agent capable of binding to the polypeptide, wherein the binding agent comprises a coding tag that comprises identifying information regarding the binding agent;

c. transferring the identifying information from the coding tag to the recording tag to generate an extended recording tag; and

d. analyzing the extended recording tag, thereby analyzing the polypeptide.

In some embodiments of the conjugate of the polypeptide and a recording tag, L² is a linking group comprising C₁₋₁₂ alkylene. In some embodiments, L² is —O—Z-, wherein Z is C₂—C₁₂ alkylene. In some embodiments, -L²-M is —O—Z-CC, wherein Z is C₂—C₁₂ alkylene, CC is a biorthogonal handle, herein the CC is configured to connect the conjugate to the recording tag. In some embodiments, CC comprises a click chemistry reactant described herein.

In yet another embodiment, the present disclosure provides a method for C-terminal N-functionalization of a polypeptide, comprising reacting the peptide of Formula:

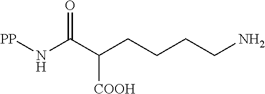

or a salt thereof, wherein PP is a polypeptide, with an acylating agent of Formula (I):

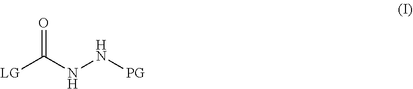

wherein LG is a leaving group, and PG is a nitrogen protecting group, in a buffer having a pH about 7.5 to about 11.5, to obtain a modified polypeptide comprising at least one modified lysine residue, and having the formula:

PP—(CH₂)₄—NH—C(=O)—NH—NH—PG,

wherein PP is the polypeptide;

—(CH₂)₄—NH— is the side chain of a lysine residue in the polypeptide; and PG is the nitrogen protecting group.

Various embodiments apply equally to the aspects provided herein but will for the sake of brevity be recited only once. Thus, various of the following embodiments apply equally to aspects recited below.

In some embodiments, the modified polypeptide comprises an N-terminal lysine residue that is modified as described herein. In some embodiments, the modified polypeptide comprises an internal lysine residue that is modified as described herein. In some embodiments, the modified polypeptide comprises a C-terminal lysine residue that is modified as described herein.

In some embodiments, the modified lysine residue of the modified polypeptide is a C-terminal amino acid residue. In some embodiments, the polypeptide is obtained from a biological sample by fragmenting proteins present in the biological sample with a site-specific protease, such as Trypsin or Lys-C, before the reacting step with the acylating agent of Formula (I). In some embodiments, the site-specific protease fragments proteins present in the biological sample in such way to generate a plurality of polypeptides, including the polypeptide to be reacted or analyzed, each having a Lysine (Lys) residue as a C-terminal amino acid residue.

In some embodiments, the amino group of the N-terminal amino acid residue of the polypeptide is protected or blocked before the reacting step with the acylating agent of Formula (I). In some embodiments, the N-terminal amino acid residue of the polypeptide is attached to the solid support before the reacting step with the acylating agent of Formula (I).

In preffered embodiments, the solid support is a bead, microparticle or microsphere.

In some embodiments, the disclosed methods provide a modified polypeptide comprising at least one modified lysine residue, and having the formula: PP—(CH₂)₄—NH—C(=O)—NH—NH—PG, wherein PP is the polypeptide; —(CH₂)₄—NH— is the side chain of a lysine residue in the polypeptide; and PG is the nitrogen protecting group.

In some embodiments of any of the acylating agent Formula (I) used herein, for example, the acylating agent used in the method of modifying a polypeptide, the method for C-terminal N-functionalization of a polypeptide, the method of preparing a conjugate, or the method of analyzing a polypeptide, PG is the nitrogen protecting group and can be selected from the list consisting of a carbamate group, sulfonamide group and acyl group. In some embodiments, PG is the nitrogen protecting group selected from the list consisting of an ester group, sulfonyl group and acyl group. In some embodiments, —NH-PG is selected from the list consisting of a carbamate group, sulfonamide group and amide group. In some preffered embodiments, the nitrogen protecting group is selected from a tert-butoxy carbamate, a fluorenylmethoxy carbamate, a dimethylaminooxy carbamate, a 2-(trimethylsilyl)ethoxycarbamate, a 2-(trimethylsilyl)ethanesulfonamide, and a picoloyl amide. In some preffered embodiments, the nitrogen protecting group, together with the nitrogen being protected, form a group selected from the group consisting of a tert-butoxy carbamate, a fluorenylmethoxy carbamate, a dimethylaminooxy carbamate, a 2-(trimethylsilyl)ethoxycarbamate, a 2-(trimethylsilyl)ethanesulfonamide, and a picoloyl amide.

In some embodiments of any of the acylating agent Formula (I) used herein, for example, the acylating agent used in the method of modifying a polypeptide, the method for C-terminal N-functionalization of a polypeptide, the method of preparing a conjugate, or the method of analyzing a polypeptide, LG can be a phenoxy group wherein the phenyl ring of the phenoxy group is optionally substituted with up to four independently selected electron-withdrawing substituents. In some preffered embodiments, LG is phenoxy substituted with one to three groups independently selected from halo, haloalkyl, haloalkoxy, nitro, and cyano. In some preffered embodiments, the phenoxy is substituted with one to three nitro groups. In some embodiments, the phenoxy is substituted with one nitro group at para or ortho position.

In some embodiments, the disclosed methods further comprise removing the nitrogen protecting group from PP—(CH₂)₄—NH—C(=O)—NH—NH—PG, to provide a modified polypeptide of the formula PP—(CH₂)₄—NH—C(=O)—NH—NH₂, wherein PP is the polypeptide, —(CH₂)₄NH— is the side chain of a lysine residue in the polypeptide, and PG is the nitrogen protecting group or H.

In some embodiments, the disclosed methods further comprise a step of contacting the modified polypeptide of the formula PP—(CH₂)₄—NH—C(=O)—NH—NH₂ with a substituted ortho-acylphenylboronic acid of the formula:

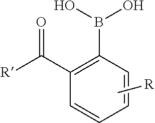

to form a diazaborine of the formula:

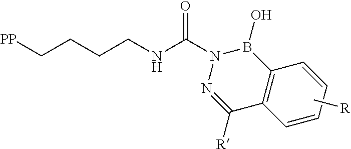

wherein

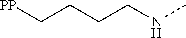

is the polypeptide connected to the acyl group on the diazaborine via a lysine residue of the polypeptide, R′ is H or C1-4 alkyl, and

R is a substituent group on the phenyl ring, which comprises a cargo moiety or a reactive functional moiety to enable connection of R to a cargo moiety.

In preferred embodiments, PG is the nitrogen protecting group. In some embodiments, PG is the nitrogen protecting group that does not require a separate step of removal before the step of contacting the modified polypeptide of the formula PP—(CH₂)₄—NH—C(=O)—NH—NH₂ with a substituted ortho-acylphenylboronic acid of the formula:

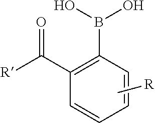

to form a diazaborine of the formula:

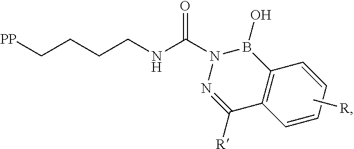

wherein

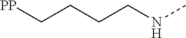

is the polypeptide connected to the acyl group on the diazaborine via a lysine residue of the polypeptide. In some embodiments, the nitrogen protecting group may be removed spontaneously. In some embodiments, the removal may be induced by a specific buffer condition used during the reaction between the modified polypeptide of the formula PP—(CH₂)₄—NH—C(=O)—NH—NH₂ with the substituted ortho-acylphenylboronic acid shown above.

In some embodiments, R is a group of the formula —L2—M, wherein L2 is a linking group.

In some embodiments, M is a cargo moiety selected from a polypeptide, a polynucleotide, and a polysaccharide.

In some embodiments, M is a biorthogonal handle for attaching R to a cargo moiety linked to a complementary biorthogonal handle. In some embodiments of the foregoing, the cargo moiety is selected from a polypeptide, a polynucleotide, and a polysaccharide.

In some embodiments, R′ is H. In other embodiments, R′ is Methyl.

In some embodiments, the —L2—M in any of the modified polypeptide or conjugate described herein can have the formula —O—Z-CC, wherein Z is C₂—C₁₂ alkylene, and CC is a bioorthogonal handle.

In some embodiments, the substituted ortho-acylphenylboronic acid (e.g., the substituted ortho-acylphenylboronic acid used in the method of modifying a polypeptide, the method for C-terminal N-functionalization of a polypeptide, the method of preparing a conjugate, or the method of analyzing a polypeptide) is of the formula:

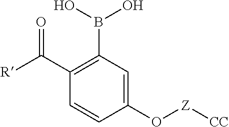

wherein Z is C₂—C₁₂ alkylene, and CC is a bioorthogonal handle. In some embodiments, CC comprises a click chemistry reactant. Suitable click functional groups may include functional groups compatible with a nucleophilic addition reaction, a cyclopropane-tetrazine reaction, a strain-promoted azide-alkyne cycloaddition (SPAAC) reaction, an alkyne hydrothiolation reaction, an alkene hydrothiolation reaction, a strain-promoted alkyne-nitrone cycloaddition (SPANC) reaction, an inverse electron-demand Diels-Alder (IED-DA) reaction, a cyanobenzothiazole condensation reaction, an aldehyde/ketone condensation reaction, and Cu(I)-catalyzed azide-alkyne cycloaddition (CuAAC) reaction. In some embodiments, the biorthogonal handle CC can comprise or be any functional group involved in click reactions. In some embodiments, such click reactions may involve (i) azido and cyclooctynyl; (ii) azido and alkynyl; (iii) tetrazine and dienophile; (iv) thiol and alkynyl; (v) cyano and amino thiol; (vi) nitrone and cyclooctynyl; or (vii) cyclooctynyl and nitrone. It should be recognized that in instances in which the biorthogonal handle CC comprises or is a click functional group, the other biorthogonal handle to which it is capable of forming a covalent bond comprises the complementary click functional group to that of the biorthogonal handle CC. For example, in some embodiments, the first biorthogonal handle CC comprises or is an azide moiety and the second biorthogonal handle CC comprises a complementary alkyne moiety, or vice versa.

In some embodiments, the substituted ortho-acylphenylboronic acid (e.g., the substituted ortho-acylphenylboronic acid used in the method of modifying a polypeptide, the method for C-terminal N-functionalization of a polypeptide, the method of preparing a conjugate, or the method of analyzing a polypeptide) is of the formula:

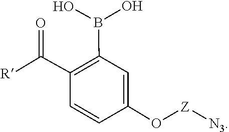

In some embodiments, the disclosed methods further comprise before contacting the polypeptide with an acylating agent of Formula (I), coupling an N-terminal amine group of an N-terminal amino acid (NTAA) residue of the polypeptide with a blocking group or to a solid support.

In some embodiments, an N-terminal amine group of an N-terminal amino acid (NTAA) residue of the polypeptide is attached to a solid support.

In some embodiments of the disclosed method, the biorthogonal handle CC in the substituted ortho-acylphenylboronic acid is a first biorthogonal handle. In some embodiments, the disclosed methods further comprise a step of contacting the disclosed conjugate attached to the first bioorthogonal handle, with a cargo moiety attached to a second bioorthogonal handle that is complementary to the first bioorthogonal handle, under conditions where the first bioorthogonal handle forms a covalent connection with the second bioorthogonal handle, thereby forming the polypeptide—cargo conjugate.

In some embodiments, the disclosed methods further comprise a step of providing the polypeptide prior to step (a), the step comprising: fragmenting proteins from a biological sample to generate a plurality of polypeptides comprising the polypeptide.

In preffered embodiments of the disclosed methods, the polypeptide analyte and anassociated nucleic acid recording tag are attached to the solid support using methods disclosed herein. In some embodiments, the method of polypeptide analysis comprises the steps of contacting the polypeptide with a binding agent capable of binding to the polypeptide, wherein the binding agent comprises a coding tag that comprises identifying information regarding the binding agent; and analyzing the polypeptide by c1) transferring the identifying information from the coding tag to the recording tag to generate an extended recording tag; and c2) analyzing the extended recording tag. These embodiments are further described in Example 5 below. In other embodiments of the disclosed methods, the method of polypeptide analysis comprises the steps of contacting the polypeptide with a binding agent capable of binding to the polypeptide, wherein the binding agent comprises a detectable label; and analyzing the polypeptide by (i) obtaining signal from detectable label upon binding of the binding agent to the polypeptide. Detectable labels include, but are not limited to, fluorophores, bioluminescent proteins, nucleic acid segments including a constant region and barcode region, or chemical tethers for linking to a nanoparticle such as a magnetic particle. In some embodiments, the methods using an optical detector configured to detect binding of the panel of different binding agents to the plurality of different polypeptide analytes attached to the solid support. Detectable labels may include several different flurophores with different patterns of excitation or emission. After binding of binding agents to the plurality of different polypeptide analytes, a plurality of signals from detectable labels of binding agents are detected and analyzed, for example, as disclosed in patent publications US 20200209255 A1, US 11549,942 B2, US 20180299460 A1, incorporated by reference herein.

The analyzing step may comprise polypeptide identification, which comprise a software tool to determine likely identities of each polypeptide analyte at certain place of the solid support from the information about which binding agents bound to that analyte. The software may utilize information about the binding characteristics of each binding agent. For example, a given binding agent may preferentially binds to certain N-terminal amino acids of polypeptide analytes attached to the solid support. Given the information about the binding characteristic of each binding agent, a database of the expected polypeptide present in the sample, the pattern of binding, the software tool may assign a probable identity to each polypeptide present in the certain place in the solid support. In cases where the binding characteristics are highly complex, an expectation maximization approach may be employed.

The following enumerated embodiments are representative of the invention:

Embodiment 1. A method to modify a polypeptide that contains at least one lysine residue, which method comprises contacting the polypeptide with an acylating agent of Formula (I):

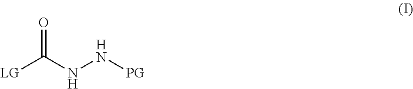

wherein:

LG is a leaving group; and

PG is a nitrogen protecting group.

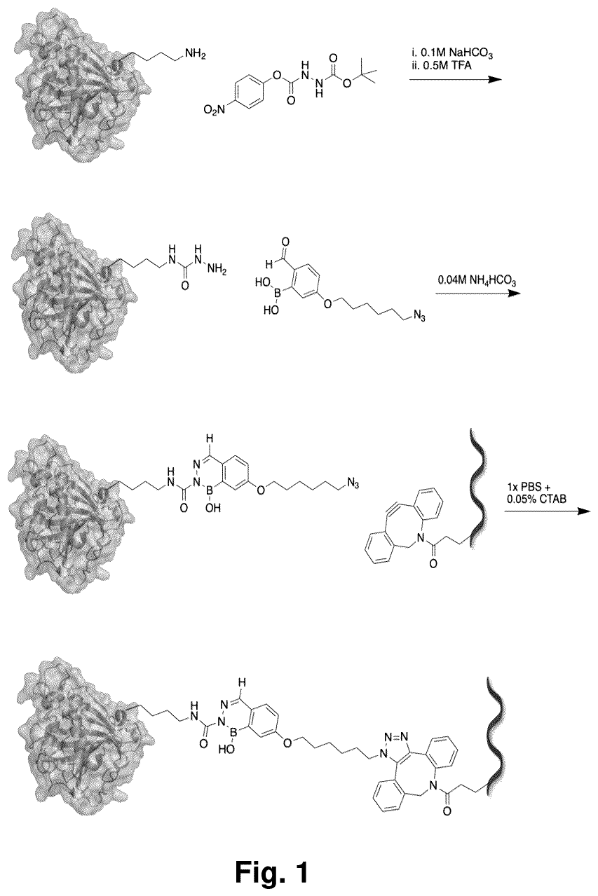

In some such embodiments, LG is a phenoxy group substituted with one or two electron withdrawing groups, such as nitro, cyano, and/or halo. Suitable examples of the nitrogen protecting group PG include t-butoxy, benzyloxy, fluorenylmethoxy, and trimethylsilylethoxy carbamates. The acylating agent depicted in FIG. 1 is a suitable example of a compound of Formula (I) for use in the methods described herein.
Embodiment 2. The method of embodiment 1, wherein the method provides a modified polypeptide comprising at least one modified lysine residue, and having the formula:

PP—(CH₂)₄—NH—C(=O)—NH—NH—PG,

wherein PP is the polypeptide;

—(CH₂)₄—NH— is the side chain of a lysine residue in the polypeptide; and PG is the nitrogen protecting group or H.

Embodiment 3. The method of embodiment 1 or 2, wherein the nitrogen protecting group is a carbamate, sulfonamide or acyl. Optionally, the method can include a further step of removing the nitrogen protecting group after modification of the polypeptide; methods for removing the protecting groups are well known.
Embodiment 4. The method of any one of the preceding embodiments, wherein the nitrogen protecting group is selected from a tert-butoxy carbamate, a fluorenylmethoxy carbamate, a dimethylaminooxy carbamate, a 2-(trimethylsilyl)ethoxycarbamate, a 2-(trimethylsilyl)ethanesulfonamide, and a picoloyl amide.
Embodiment 5. The method of any one of the preceding embodiments, wherein LG is a Phenoxy group wherein the phenyl ring of the phenoxy group is optionally substituted with up to four electron-withdrawing substituents.
Embodiment 6. The method of embodiment 5, wherein LG is phenoxy substituted with one to three groups selected from halo, haloalkyl, haloalkoxy, nitro, and cyano.
Embodiment 7. The method of embodiment 5, wherein LG is phenoxy substituted with one to three nitro groups.
Embodiment 8. The method of any one of the preceding embodiments, which further comprises removing the nitrogen protecting group from PP—(CH₂)₄—NH—C(=O)—NH—NH—PG, to provide a modified polypeptide of the formula PP—(CH₂)₄—NH—C(=O)—NH—NH₂, wherein PP is the polypeptide,

—(CH₂)₄NH— is the side chain of a lysine residue in the polypeptide, and PG is the nitrogen protecting group or H.

Embodiment 9. The method of embodiment 8 which further comprises a step of contacting the modified polypeptide of the formula PP—(CH₂)₄—NH—C(=O)—NH—NH₂ with a substituted ortho-acylphenylboronic acid of the formula:

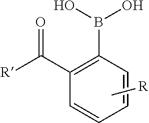

to form a diazaborine of the formula:

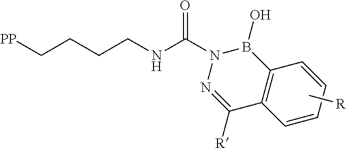

wherein

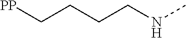

is the polypeptide connected to the acyl group on the diazaborine via a lysine residue of the polypeptide,

R′ is H or C₁₋₄ alkyl, and

R is a substituent group on the phenyl ring, which comprises a cargo moiety or a reactive functional moiety to enable connection of R to a cargo moiety.

Embodiment 10. The method of embodiment 9, wherein R is a group of the formula —L²—M,

wherein L² is a linking group, and

M is a cargo moiety selected from a polypeptide, a polynucleotide, and a polysaccharide.

A suitable example of group R in these embodiments is an azido-alkoxy group such as the side chain of the species in FIG. 1.

Embodiment 11. The method of embodiment 9, wherein R is a group of the formula

formula —L²—M,
wherein L² is a linking group, and

M is a biorthogonal handle for attaching R to a cargo moiety linked to a complementary bioorthogonal handle. L² can be an alkyl or alkoxy group, and M can be any known bioorthogonal handle described herein.

Embodiment 12. The method of any one of embodiments 1-11, wherein R′ is H or Methyl.

In some particular embodiments, R′ is H.

Embodiment 13. The method of any one of embodiments 9-12, wherein the substituted ortho-acylphenylboronic acid is of the formula:

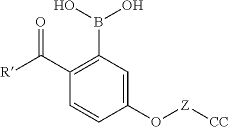

wherein Z is C₂—C₁₂ alkylene, and

CC is a bioorthogonal handle such as a click chemistry reactant. In some particular embodiments, R′ is H.

Embodiment 14. The method of embodiment 13, wherein the substituted ortho-acylphenylboronic acid is of the formula:

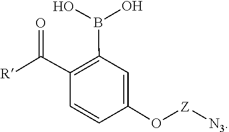

In some particular embodiments, R′ is H.
Embodiment 15. The method of any one of embodiments 1-14, further comprising, before contacting the polypeptide with an acylating agent of Formula (I), coupling an N-terminal amine group of an N-terminal amino acid (NTAA) residue of the polypeptide with a blocking group or to a solid support.
Embodiment 16. A conjugate comprising a polypeptide connected by a tether to a cargo moiety or a reactive functional moiety, wherein the tether comprises a diazaborine, and wherein the conjugate has the formula:

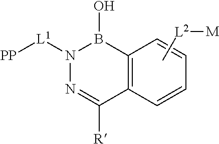

wherein:

R′ is H or C₁₋₄ alkyl;

PP is the polypeptide;

M is the cargo moiety or the reactive functional moiety; M is optionally selected from the group consisting of a polypeptide, a polynucleotide, and a polysaccharide;

L¹ is a linker connecting the diazaborine to PP; and

L² is a linker connecting the polypeptide to M.

Embodiment 17. The conjugate of embodiment 16, wherein R′ is H.
Embodiment 18. The conjugate of embodiment 16, wherein R′ is methyl.
Embodiment 19. The conjugate of embodiment 16, which is of the formula:

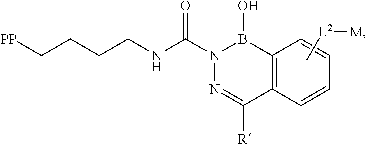

wherein

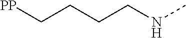

represents connection of the polypeptide to a carbonyl group on the diazaborine through a lysine residue of the polypeptide.
Embodiment 20. The conjugate of any one of embodiments 16-19, which is attached to a solid support, such as wherein an N-terminal amine group of an N-terminal amino acid (NTAA) residue of the polypeptide is coupled to the solid support.
Embodiment 21. A method for preparing a conjugate of embodiment 16 having the formula:

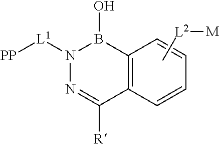

wherein the method comprises the following steps:

a. modifying a polypeptide that contains at least one lysine residue by a method which comprises contacting the polypeptide with an acylating agent of Formula (I) to attach a semicarbazide group to the at least one lysine residue to form a polypeptide semicarbazide compound, such as a method according to any one of embodiments 1-8;

b. optionally, removing any protecting group present on the semicarbazide group of the polypeptide semicarbazide compound (wherein, optionally, the nitrogen protecting group is a carbamate, sulfonamide or acyl); and

c. contacting the polypeptide semicarbazide compound with an ortho-acyl phenylboronic acid of the formula:

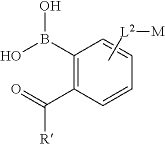

wherein L² is a linking group and M is a cargo moiety or a reactive functional moiety that is configured to connect the conjugate to a cargo moiety, under conditions where the semicarbazide group reacts with the ortho-acyl phenylboronic acid to form a diazaborine to provide the conjugate;

d. optionally, when M is a reactive functional moiety, using the reactive functional moiety to connect the conjugate to a cargo moiety. In this embodiment, the final product comprises M that is a cargo moiety.

Embodiment 22. The method of embodiment 21, wherein M is a cargo moiety.
Embodiment 23. The method of embodiment 21, wherein M is a reactive functional moiety that can be used to attach the linker L² to a cargo moiety.
Embodiment 24. The method of embodiment 23, wherein the reactive functional moiety is a first bioorthogonal handle.
Embodiment 25. The method of embodiment 24, further comprising a step of contacting the conjugate attached to the first bioorthogonal handle, with a cargo moiety attached to a second bioorthogonal handle that is complementary to the first bioorthogonal handle,
under conditions where the first bioorthogonal handle forms a covalent connection with the second bioorthogonal handle,
thereby forming the polypeptide—cargo conjugate.
   Embodiment 26. The method of any one of embodiments 21-25, wherein R′ is H or methyl.
   Embodiment 27. The method of any one of embodiments 21-26, further comprising a step of providing the polypeptide prior to step (a), the step comprising: fragmenting proteins from a biological sample to generate a plurality of polypeptides comprising the polypeptide.
   Embodiment 28. The method of embodiment 27, wherein step of providing the polypeptide further comprises coupling an N-terminal amine group of an N-terminal amino acid (NTAA) residue of the polypeptide to a solid support.
   Embodiment 29. A method of analyzing a polypeptide comprising at least one lysine residue, the method comprising the steps of:

a. providing a conjugate of the polypeptide and a recording tag, the conjugate attached to a solid support, wherein the recording tag comprises a polynucleotide that is conjugated to the polypeptide according to the following steps:
(i) contacting the polypeptide with an acylating agent of Formula (I)

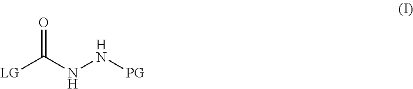

to attach a semicarbazide group to the at least one lysine residue to form a polypeptide semicarbazide compound, e.g., by a method according to any one of embodiments 1-8;
(ii) optionally, removing the nitrogen protecting group (PG) present on the semicarbazide group of the polypeptide semicarbazide compound; and
(iii) contacting the polypeptide semicarbazide compound with an ortho-acyl phenylboronic acid of the formula:

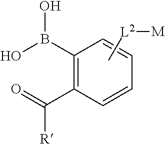

wherein L² is a linking group and M is the recording tag or a reactive functional moiety that is configured to connect the conjugate to the recording tag, under conditions where the semicarbazide group reacts with the ortho-acyl phenylboronic acid to form a diazaborine to provide the conjugate;
(iv) optionally, when M is the reactive functional moiety, using the reactive functional moiety to connect the conjugate to the recording tag;
(v) attaching the polypeptide or the conjugate to the solid support before or after any one of the steps (i)-(iv);

b. contacting the polypeptide of the conjugate with a binding agent capable of binding to the polypeptide, wherein the binding agent comprises a coding tag that comprises identifying information regarding the binding agent;

c. transferring the identifying information from the coding tag to the recording tag to generate an extended recording tag; and

d. analyzing the extended recording tag, thereby analyzing the polypeptide.

Embodiment 30. The method of embodiment 29, wherein analyzing the polypeptide comprises identifying at least partial amino acid sequence of the polypeptide.
Embodiment 31. A conjugate having a polypeptide connected via a lysine residue of the polypeptide to a diazaborine, wherein the conjugate has the formula:

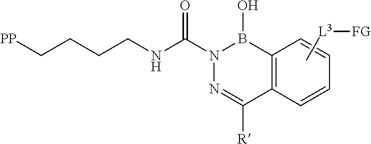

wherein:

R′ is H or C₁₋₄ alkyl;

PP is the polypeptide;

FG is a reactive functional moiety suitable for attaching the conjugate to a cargo moiety; and

L³ is a linker connecting the diazaborine to FG.

Embodiment 32. A method of analyzing a polypeptide comprising at least one lysine residue, the method comprising the steps of:

a. providing a conjugate of the polypeptide and a recording tag on a solid support, wherein the recording tag comprises a polynucleotide that is conjugated to the polypeptide according to the following steps:
(i) contacting the polypeptide with an acylating agent of Formula (I)

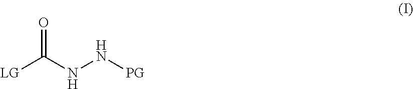

wherein: LG is a leaving group; and PG is a nitrogen protecting group, to attach a semicarbazide group to the at least one lysine residue to form a polypeptide semicarbazide compound;
(ii) optionally, removing any protecting group present on the semicarbazide group of the polypeptide semicarbazide compound; and
(iii) contacting the polypeptide semicarbazide compound with an ortho-acyl phenylboronic acid of the formula:

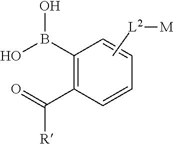

wherein L² is a linking group and M is the recording tag or a reactive functional moiety that is configured to connect the conjugate to the recording tag, under conditions where the semicarbazide group reacts with the ortho-acyl phenylboronic acid to form a diazaborine to provide the conjugate;
(iv) optionally, when M is the reactive functional moiety, using the reactive functional moiety to connect the conjugate to the recording tag;
(v) attaching the polypeptide or the conjugate to the solid support before or after any one of the steps (i)-(iv);

b. contacting the polypeptide with a binding agent capable of binding to the polypeptide, wherein the binding agent comprises (i) a coding tag that comprises identifying information regarding the binding agent; or (ii) a detectable label; and

c. analyzing the polypeptide by (i) obtaining signal from detectable label upon binding of the binding agent to the polypeptide; or (ii) c1) transferring the identifying information from the coding tag to the recording tag to generate an extended recording tag; and c2) analyzing the extended recording tag.

Embodiment 33. The method of embodiment 32, wherein the nitrogen protecting group is a carbamate, sulfonamide or acyl.
Embodiment 34. The method of embodiment 32, wherein analyzing the polypeptide comprises identifying an amino acid sequence of the polypeptide.

### EXAMPLES

The following examples are offered to illustrate but not to limit the methods, compositions, and uses provided herein. Certain aspects of the present invention, including, but not limited to, embodiments for ProteoCode™ polypeptide analysis assay, methods for attachment of nucleotide-polypeptides conjugate to a support, methods of making nucleotide-polypeptide conjugates, methods of generating barcodes, methods of generating binding agents for polypeptide analysis, methods of analyzing extended recording tags to analyze a component of a polypeptide analyte were disclosed in earlier published applications US 2019/0145982 A1, US 2020/0348308 A1, US 2020/0348307 A1, US 2021/0214701 A1, US 2022/0049246 A1, US 2022/0283175 A1, and US 2022/0144885 A1, the contents of which are incorporated herein by reference in their entireties.

In these examples, (2-formyl-5-(6-azidohexyloxy)phenyl)boronic acid is used to demonstrate diazaborine conjugation at the protein-level in several varieties. First in-solution, with the azide-containing FPBA then addition of DBCO-oligo to form the polypeptide-oligo conjugate. These reactions were also subjected to trypsinization (with or without 8M urea for denaturation, which did not affect efficiency of conjugation), showing successful conjugations and digestion. Second, forming the semicarbazide-labeled polypeptides, then conjugation to FPBA-oligo in solution. Lastly, using semicarbazide-labeled polypeptides and pulling them down on FPBA-Recording Tag bead successfully.

Example 1. Assessment of reaction conditions for lysine residue modification for a model polypeptide.

The reaction workfow for the reactions described in this Example is shown in FIG. 1.

The procedure begins with treating a polypeptide-containing sample with aqueous 0.1M NaHCO 3 (pH 8.3) and a semicarbazide transfer reagent (1-(tert-butyl) 2-(4-nitrophenyl) hydrazine-1,2-dicarboxylate) (total volume 100 uL) for lh at 37° C. For the disclosed modification reactions, acceptable buffer range was pH 7.5-11.5, and acceptable molarity of buffers was not typically over 200 mM. Other suitable buffers include non-primary or secondary amine-bearing buffers, such as sodium or potassium phosphate, sodium borate, sodium or potassium carbonate, MOPS, PIPES, HEPES, triethylammonium acetate, N-methyl- or N-ethylmorpholinium acetate. Upon completion, 50 uL of 2.5M trifluoroacetic acid or trichloroacetic acid was added to the solution at 65° C. for lh to perform three tasks: quench the semicarbazide transfer reaction, deprotect the Boc protecting group(s), and aid in precipitation of the functionalized polypeptide(s). Alternative ways to quench the semicarbazide transfer reaction are also possible, such as separation of modified polypeptides from low molecular weight reaction products by filtering. After 1 h, the tube was placed on ice and then diluted with 450 uL of cold 100% alcohol (for example, ethanol or isopropanol). The sample was vortexed and centrifuged for 3 minutes at 10,000 rpm. The supernatant was removed and discarded. To this, 600 uL of cold 75% alcohol was added to the pellet and it was resuspended, and centrifuged. The process was repeated a total of 3 times to aid in further precipitation and removal of semicarbazide (SC) transfer reagent and its reaction components. Finally, the pellet was resuspended in 0.1M MOPS buffer (pH 7.5) containing 0.1% Tween 20 and was heated at 60° C. for 30 minutes to re-solubilize the functionalized polypeptides.

At this step the sample contained polypeptides with semicarbazide modified free amines (lysines and N-termini) ready for conjugation to 2-formylphenylboronic acid (FPBA) or 2-acetylphenylboronic acid conjugated to a reactive handle. Semicarbazide-FPBA reaction has rapid kinetics (krel>10³ M⁻¹s⁻¹)

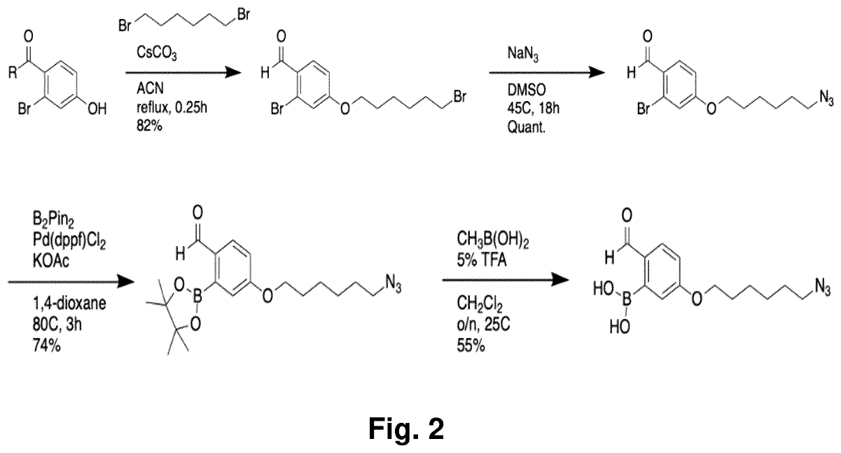

FPBA conjugated to azide moiety was added to SC-modified polypeptide solution, and the mixture was incubated for 1 h at 37° C. to facilitate the conjugation between the SC-modified polypeptide and FPBA, which results in a polypeptide conjugated to the azide moiety through irreversible diazaborine moiety. Different ratios of SC-polypeptide to FPBA-N₃ were employed, ranging from 10000:1 to 1:1. Exemplary synthesis of FPBA-N₃ is shown in FIG. 2.

Finally, the modified polypeptide was conjugated to an oligonucleotide via addition of DBCO-oligonucleotide, allowing azide-DBCO reaction to occur in PBS and in the presence of 0.05% of CTAB detergent (using CTAB as a surfactant catalyst for DBCO-N₃ click chemistry reaction results in about 100 times rate enhancement).

In this and the following examples, (2-formyl-5-(6-azidohexyloxy)phenyl)boronic acid is used to demonstrate diazaborine conjugation to lysine residues on polypeptides in several varieties. First, in solution reaction between semicarbazide-modified polypeptide and the azide-containing FPBA was explored, followed by addition of DBCO-oligonucleotide to form the polypeptide-oligonucleotide conjugate. These reactions were also subjected to trypsinization (with or without addition of urea to denature polypeptides), showing successful conjugations and digestion. Second, in solution reactions between semicarbazide-modified polypeptide and the FPBA-oligonucleotide fusions were explored, producing polypeptide- oligonucleotide conjugates. Third, reactions between semicarbazide-modified polypeptide and the FPBA-oligonucleotide fusions attached to solid support (beads) were explored, producing polypeptide-oligonucleotide conjugates immobilized on the beads (see Example 2).

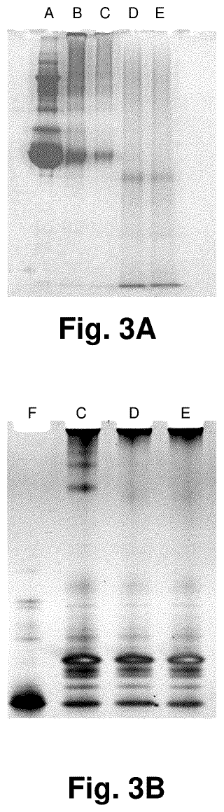

Exemplary conjugation reactions showing conjugation efficiency under different conditions are shown in FIG. 3A-3B. The samples analyzed in FIG. 3A-3B were as follows: (A)—serum stock (protein only); (B)—SC-modified serum proteins conjugated with FPBA-N₃; (C)—SC-modified serum proteins conjugated with FPBA-DNA; (D)—SC-modified serum proteins treated with 1.4M Urea and Trypsin (in 0.1M Tris at pH=8), then conjugated with FPBA-DNA; (E)—SC-modified serum proteins treated with Trypsin (in 0.1M Tris at pH=8), then conjugated with FPBA-DNA; (F)—DBCO-DNA only. FIG. 3A shows analysis of the corresponding samples using gel electrophoresis (200 V, 50 min) in 16% Tris-Gly Protein Gel, whereas FIG. 3B shows analysis of the corresponding samples using gel electrophoresis (200 V, 20 min) in 15% TBU DNA Gel. Conjugation efficiency was monitored by appearance of high molecular weight (HMW) smears. As expected, trypsinization effectively reduced molecular weight of conjugated fractions. Data from both protein and DNA gels confirm efficient in-solution formation of protein-DNA conjugates.

Example 2. Modification of lysine residues of serum polypeptides with semicarbazide, and preparing polypeptide conjugates with formylphenylboronic acid-labeled oligonucleotides using the Boc-protected SC transfer reagent.

For this example, serum was obtained from pooled human sources that have been heat inactivated and screened for potential blood-borne pathogens. These serum samples were not depleted, meaning the majority of the polypeptide content was albumin and immunoglobulins.

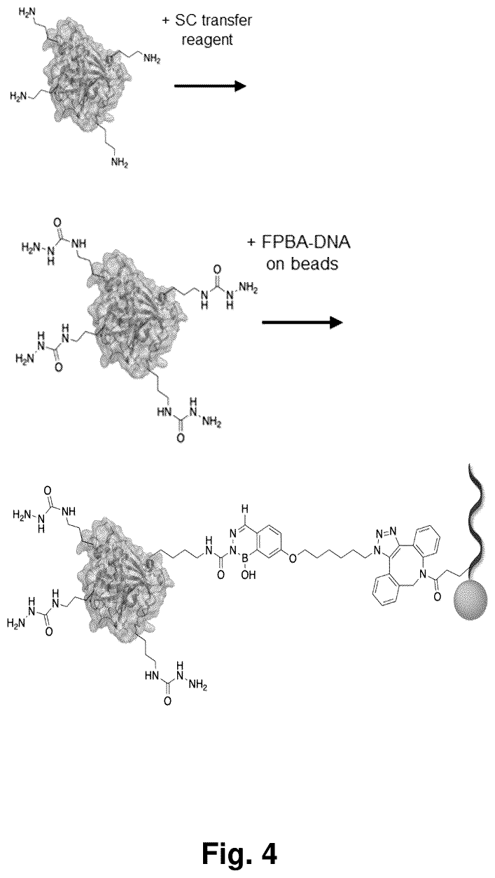

The reaction workfow for the described conjugation reactions is shown in FIG. 4. To a 1.5 mL microcentrifuge tube, a 20 uL aliquot of a pooled human serum polypeptide sample (50mg/mL) was added to 80 uL of 0.1M NaHCO₃ (pH 8.3). For the disclosed modification reactions, acceptable buffer range was pH 7.5-11.5, and acceptable molarity of buffers was not typically over 200 mM. Other suitable buffers include non-primary or secondary amine-bearing buffers, such as sodium or potassium phosphate, sodium borate, sodium or potassium carbonate, MOPS, PIPES, HEPES, triethylammonium acetate, N-methyl- or N-ethylmorpholinium acetate. To this, 20 uL of 200 mM semicarbazide transfer reagent (1-(tert-butyl)-2-(4-nitrophenyl) hydrazine-1,2-dicarboxylate) in DMSO was added and the solution was mixed vigorously (resulting in a yellow solution) and placed on a thermomixer at 37° C. for 1-2 hours. Upon completion, the solution was diluted with 80 uL of 2.5M trifluoroacetic acid (TFA; aq.) or 2.5M trichloroacetic acid (TCA; aq.) and placed on the thermomixer for lh at 60° C. Upon completion, the solution was cloudy and colorless. To this, 600 uL of 100% isopropanol was added and the tube was put on ice for 20 minutes. Then the tube was centrifuged at 12,000 rpm for 3 minutes and the supernatant was removed by pipetting. The polypeptide pellet was resuspended in 75% isopropanol (aq.) and centrifuged at 12,000 rpm. The resulting polypeptide pellet was separated from the supernatant by pipetting and this process was repeated 3×. Finally, the pellet was resuspended in 0.1M MOPS buffer (pH 7.5) containing 0.1% Tween 20 and was heated at 60° C. for 30 minutes to re-solubilize the functionalized polypeptides.

2-formylphenylboronic acid-containing beads were synthesized by treating commercially available NHS-ester sepharose beads with a ratio of mTet-amine: mPEG-amine for a desired functional density. Then, the beads were treated with heterobifunctional TCO-DNA hairpin-DBCO to affix the hairpin to the beads, washed with 1M NaCl (2×), 1× PBST (2×) and resuspended in PBST to a bead concentration of 1000-4000 beads/uL. Upon completion, the DNA hairpin-DBCO beads were treated with 20 mM 2-FPBA-C6-N₃ in DMSO in 3:1 PBST:DMSO mixture (5 mM effective 2-FPBA-C6-N₃ concentration) for 18 hours at 25° C., washed with 1:1 acetonitrile:water (2×), 1M NaCl (2×), and 1× PBST (2x) and resuspending in PBST at a beads concentration of 2000 beads/uL. The SC-functionalized polypeptide solution was then added to a solution of sepharose beads affixed with oligonucleotide hairpin-containing 2-formylphenylboronic acid in 0.05M MOPS (pH=7.5). The oligonucleotide hairpin contains a nucleic acid recording tag suitable for use in downstream aasays, such as ProteoCode™ assay (see Example 4). The mixture was incubated for 1 h at 37° C. to facilitate the conjugation between the semicarbazide-labeled polypeptide and the 2-formylphenylboronic acid-oligonucleotide hairpin. Different ratios of SC-polypeptides to FPBA-oligonucleotide were employed, ranging from 10000:1 to 1:1. Typically, SC-polypeptide concentrations in the mixture were about 5 mg/mL; and concentrations of FPBA affixed on oligonucleotide hairpin were about 2 pmol.

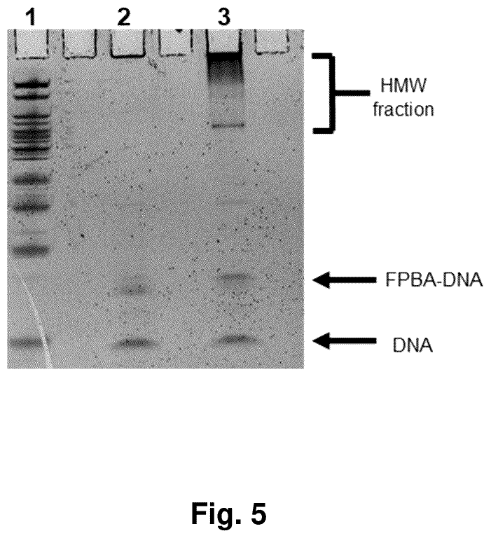

To evaluate efficiency of the conjugation, the resulting conjugate mixture was subjected to a restriction enzyme digestion and an aliquot was loaded onto a 15% TBE gel and stained with SYBR Gold stain for nucleic acids. FIG. 5 shows ssDNA ladder in lane (1), oligonucleotide-only sample (before conjugation with the semicarbazide-labeled polypeptides) in lane (2), and oligonucleotide-polypeptides conjugates after the FPBA functionalization and SC-polypeptides conjugation in lane (3). Formation of high molecular weight smear in lane 3 demonstrates successful pulldown of semicarbazide-labeled polypeptides from solution onto the FPBA-functionalized beads.

Example 3. Modification of lysine residues of serum polypeptides with semicarbazide, and preparing polypeptide conjugates with formylphenylboronic acid-labeled oligonucleotides using the Teoc-protected SC transfer reagent.

For workflow with Teoc (trimethylsilylethoxycarbonyl) as the protecting group, the following conditions were used. To a 1.5 mL microcentrifuge tube, a 20 uL aliquot of a pooled human serum polypeptide sample (50 mg/mL) was added to 80 uL of 0.1M NaHCO₃ (pH 8.3). For the disclosed modification reactions, acceptable buffer range was pH 7.5-11.5; and other suitable buffers include non-primary or secondary amine-bearing buffers, such as phosphate, borate, carbonate, MOPS, and others. To this, 20 uL of 200 mM semicarbazide transfer reagent (1-(Trimethylsilyl)ethoxycarbonyl-2-(4-nitrophenyl) hydrazine-1,2-dicarboxylate) in DMSO was added and the solution was mixed vigorously (resulting in a yellow solution) and placed on a thermomixer at 37° C. for 1-2h. Upon completion, the solution was diluted with 300uL of water and the 400uL of polypeptide solution was added to a 3 kDa molecular weight cut-off (MWCO) spin filter. The solution containing MWCO filter was placed into a 2 mL microcentrifuge tube and spun for 5 minutes at 14×1000 g. About 300 uL was added and the MWCO filter was spun again; this was repeated 3×. Upon completion, about 60 uL remaining contained the modified polypeptide solution. To this solution, the deprotection buffer comprised of 300 mM potassium fluoride (KF) supplemented with 30 mM 18-crown-6 in acetonitrile:PBS mixture (or other compatible polar, organic solvent) was added and incubated at 60° C. for 2h to remove the Teoc group and provide the semicarbazide lysine. The solution was then applied to FPBA-modified DNA hairpin beads prepared as described in Example 1 to affix the semicarbazide-modified polypeptides to the beads.

Example 4. Workflow for N-terminal peptide capture, SC modification and release.

This example demonstrates exemplary workflow for lysine semicarbazide modification of peptides on a solid support. After release, SC-peptides are conjugated with formylphenylboronic acid-labeled oligonucleotides, and are ready for the ProteoCode TM assay.

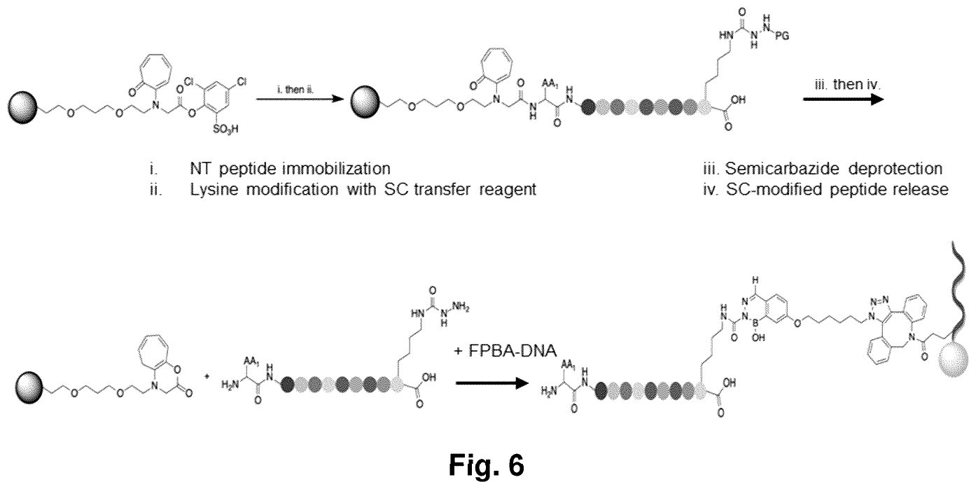

A polypeptide-containing sample comprised of 5 mg/mL native protein in a non-primary amine-based buffer, was denatured using 8M urea (or other denaturant like 2% SDS, 6M guanidine, etc.), reduced by TCEP, and alkylated at cysteines with iodoacetamide (or other cysteine capping agent like iodoacetic acid). The solution was then digested using trypsin, Lys-C, or a combination of the two proteases. This peptide solution was then applied to immobilization resin comprised of a reactive handle capable of pH-driven selective N-terminal reaction. This and the following steps are illustrated in FIG. 6. In this example, a N-tropolone-modified glycyl dichlorosulfonic acid phenylate ester is affixed to agarose beads through a pegylated linkage. Under mild pH 8.3 conditions (0.05M NaHCO₃ (aq.)) the peptides are immobilized onto the beads by their N-termini. The beads are washed using (2×; 100 uL) 1M NaCl and (2×; 100 uL) PBST (1× PBS+0.1% tween 20) to remove any non-specifically bound peptides to the surface and the beads are resuspended in 0.05M NaHCO₃. The beads are then treated with the semicarbazide transfer reagent (20 uL of 200 mM in DMSO; 33 mM effective concentration) and reacted on a ThermoMixer at 60° C. for 1 h shaking at 850 rpm. Upon completion, the beads are washed similarly as stated above ((2×; 100 uL) 1M NaCl and (2×; 100 uL) PBST) and are resuspended in the SC deprotecting buffer (for Teoc PG group: 300 mM potassium fluoride (KF) with 30 mM 18-crown-6 in acetonitrile:PBS mixture or other compatible polar, organic solvent) and incubated for 2 h at 60° C. to remove the protecting groups from the semicarbazide lysines. Upon completion, the deprotecting buffer is removed from the beads and the beads are resuspended in the release buffer, a mildly acidic methanol:water solution containing 500 mM trifluoroacetic acid (TFA). The peptides are released into solution and the solution's pH is adjusted to pH 8 by 1M NaHCO₃ solution. The mildly basic peptide solution is then applied to 2-formylphenylboronic acid (FPBA) modified DNA hairpin beads (prepared as described in Example 1) and incubated for 3 h to conjugate the C-terminal lysine-semicarbazide to the FPBA-DNA hairpin beads.

Alternatively, if the SC protecting group is Boc, the deprotecting buffer and the release buffer are one in the same and the deprotection of the SC lysine and the release of the peptides can occur simultaneously. The pH adjustment with 1M NaHCO₃ and conjugation to the FPBA-DNA hairpin beads remain the same.

Example 5. Analysis of peptide-polynucleotide conjugated immobilized on a solid support by the ProteoCode™ assay.

This example demonstrates an exemplary workflow used for analysis of peptide-polynucleotide conjugates prepared as described in the previous Examples using targeting lysine residues of protein analytes with the disclosed methods. This example describes assessing peptides' sequence information by a ProteoCode™ assay which utilizes generation of encoded DNA librares.

After the peptide-DNA conjugates prepared using the exemplary workflow described above were immobilized on a solid support, ProteoCode™ peptide analysis assay was performed as disclosed in US published patent applications US 20190145982 A1 and US 20210214701 A1, which are incorporated herein by reference in their entireties. In the assay, N-terminal amino acid (NTAA) residues of peptides from the peptide-DNA conjugates joined to the solid support (peptides with associated DNA recording tags) were functionalized by an N-terminal modification specific for recognizing binding agents. The immobilized and functionalized peptide-DNA conjugates were contacted with binding agents each conjugated with a nucleic acid coding tag containing identifying information regarding the associated binding agent. Binding agents configured to recognize chemically modified N-terminal amino acid (NTAA) residues used herein were disclosed in U.S. patent application Ser. No. 17/539,033, filed on Nov. 30, 2021; and in US 2022/0283175 A1, which are incorporated herein by reference in their entireties. Binding agents were used simultaneously as a set, and altogether have specificity for most of the modified NTAA residues. If a binding agent binds its cognate modified NTAA residue of the peptide, and affinity of the binding agent to the immobilized peptide is strong enough (typically, Kd should be less than 500 nM, and preferably, less than 200 nM), the coding tag associated with the binding agent and the recording tag associated with the peptide form hybridization complex via hybridization of the corresponding spacer regions to allow transfer of identifying information from the coding tag to the recording tag via a primer extension reaction (referred to as the “encoding reaction”), generating extended recording tag.

In some embodiments of the disclosed methods, following binding of the binding agent to the functionalized NTAA of the immobilized peptide and the encoding reaction, the functionalized NTAA is cleaved to expose a new NTAA residue of the immobilized peptide. In preferred embodiments of the disclosed methods, cleaving the functionalized NTAA residue of the peptide is done by an engineered enzyme, which is configured to cleave a peptide bond between an N-terminal functionalized amino acid residue and a penultimate terminal amino acid residue of a polypeptide, wherein the engineered cleavase is derived from a dipeptidyl aminopeptidase. Exemplary engineered cleavase enzymes capable of cleaving specific functionalized NTAA residues are disclosed in U.S. Pat. No. 11,427,814 B2, which is incorporated herein by reference in its entirety.

Following the cleavage of the functionalized NTAA from the immobilized peptide, the encoding reaction is repeated one or more times, comprising: functionalizing the newly exposed NTAA residue; binding of the binding agent with a coding tag to the newly functionalized NTAA of the immobilized peptide; and following binding, transferring identifying information from the coding tag to the extended recording tag associated with the immobilized peptide.

Three cycles of encoding (information transfer from coding tags to recording tags) with two elimination cycles in between are performed. Elimination of the NTAA exposes a new NTAA available for recognition by a binding agent provided in the next cycle. Sequencing of extended recording tags after one or more encoding cycles is used to identify binding agent(s) that was(were) bound to the immobilized peptide, providing structural information regarding the immobilized peptide. Estimating fractions of the recording tags being extended (encoded) during primer extension reaction provides estimates of efficiency of the encoding reactions, which directly correlates with binding affinity of the binder to the peptide. After completion of the binding, encoding, functionalization and elimination cycle(s), the extended recording tags are capped with an adapter sequence, subjected to PCR amplification, and analyzed by next-generation sequencing (NGS). In summary, peptides from biological samples modified using semicarbazide transfer reagent, and conjugated to nucleic acid (DNA) recording tags via FPBA conjugation (diazaborine formation) according to the exemplary workflow shown in FIG. 4 or FIG. 6 can be successfully analyzed by the ProteoCode TM peptide analysis assay.

Using the methods in these examples and general knowledge in the field, a wide array of conjugation reactions and conjugates of the invention can be practiced with various reactive handles, target molecules, detectable labels, and binding agents.

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

1. A method to modify a polypeptide that contains at least one lysine residue, which method comprises contacting the polypeptide with an acylating agent of Formula (I): 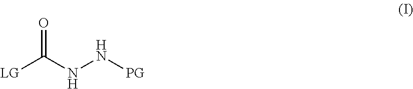
wherein:
LG is a leaving group; and
PG is a nitrogen protecting group.

2. The method of claim 1, wherein the method provides a modified polypeptide comprising at least one modified lysine residue, and having the formula:
PP—(CH₂)₄—NH—C(=O)—NH—NH—PG, wherein PP is the polypeptide;
—(CH₂)₄—NH— is the side chain of a lysine residue in the polypeptide;
and PG is the nitrogen protecting group.

3. The method of claim 1, wherein the nitrogen protecting group is a carbamate, sulfonamide or acyl.

4. The method of claim 1, wherein LG is a phenoxy group wherein the phenyl ring of the phenoxy group is optionally substituted with up to four independently selected electron-withdrawing substituents.

5. The method of claim 1, which further comprises removing the nitrogen protecting group from PP—(CH₂)₄—NH—C(=O)—NH—NH—PG, to provide a modified polypeptide of the formula PP—(CH₂)₄—NH—C(=O)—NH—NH₂,
wherein PP is the polypeptide, —(CH₂)₄NH— is the side chain of a lysine residue in the polypeptide, and PG is the nitrogen protecting group.

6. The method of claim 5, which further comprises a step of contacting the modified polypeptide of the formula PP—(CH₂)₄—NH—C(=O)—NH—NH₂ with a substituted ortho-acylphenylboronic acid of the formula: 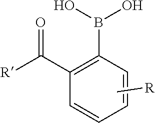
to form a diazaborine of the formula: 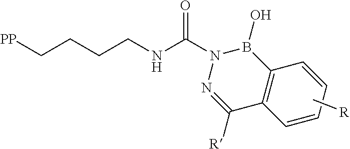
wherein 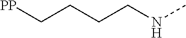
is the polypeptide connected to the acyl group on the diazaborine via a lysine residue of the polypeptide,
R′ is H or C₁₋₄ alkyl, and
R is a substituent group on the phenyl ring, which comprises a cargo moiety or a reactive functional moiety to enable connection of R to a cargo moiety.

7. The method of claim 6, wherein R is a group of the formula—L²-M,
wherein L² is a linking group, and
M is i) a cargo moiety, or ii) a biorthogonal handle for attaching R to a cargo moiety linked to a complementary biorthogonal handle; wherein the cargo moiety is selected from the group consisting of a polypeptide, a polynucleotide, and a polysaccharide.

8. The method of claim 6, wherein the substituted ortho-acylphenylboronic acid is of the formula: 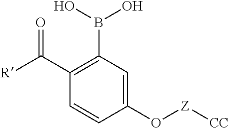
wherein Z is C₂—C₁₂ alkylene, and CC is a bioorthogonal handle such as a click chemistry reactant.

9. A conjugate comprising a polypeptide connected by a tether to a cargo moiety or a reactive functional moiety, wherein the tether comprises a diazaborine, and wherein the conjugate has the formula: 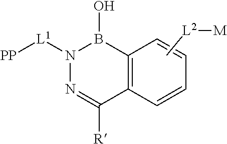
wherein:
R′ is H or C₁₋₄ alkyl;
PP is the polypeptide;
M is the cargo moiety or the reactive functional moiety;
L¹ is a linker connecting the tether to PP; and
L² is a linker connecting the tether to M.

10. The conjugate of claim 9, wherein R′ is H or methyl.

11. The conjugate of claim 9, which is of the formula: 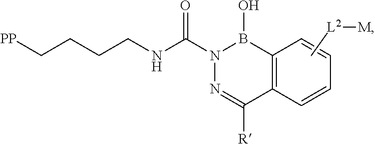
wherein 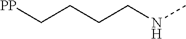
is connection of the polypeptide to a carbonyl group on the diazaborine through a lysine residue of the polypeptide.

12. The conjugate of claim 9, which is attached to a solid support.

13. The conjugate of claim 9, wherein M is the cargo moiety selected from the group consisting of a polypeptide, a polynucleotide, and a polysaccharide.

14. A method for preparing a conjugate having the formula: 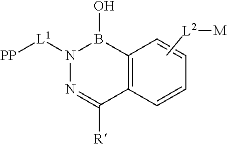
wherein R′ is H or C1-4 alkyl;
PP is a polypeptide, which is connected by a tether to M, wherein the tether comprises a diazaborine;
M is a cargo moiety or a reactive functional moiety that is configured to connect the conjugate to a cargo moiety;
L¹ is a linker connecting the tether to PP; and L² is a linker connecting the tether to M;
the method comprises the following steps:
a. modifying a polypeptide that contains at least one lysine residue by a method which comprises contacting the polypeptide with an acylating agent of Formula (I): 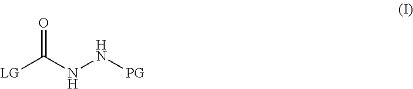
wherein:
LG is a leaving group; and PG is a nitrogen protecting group, to attach a semicarbazide group to the at least one lysine residue to form a polypeptide semicarbazide compound;
b. optionally, removing the nitrogen protecting group present on the semicarbazide group of the polypeptide semicarbazide compound; and
c. contacting the polypeptide semicarbazide compound with an ortho-acyl phenylboronic acid of the formula: 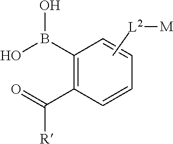
under conditions where the semicarbazide group reacts with the ortho-acyl phenylboronic acid to form a diazaborine to provide the conjugate.

15. The method of claim 14, wherein the nitrogen protecting group is a carbamate, sulfonamide or acyl.

16. The method of claim 14, further comprising a step of providing the polypeptide prior to step (a), the step comprising: fragmenting proteins from a biological sample to generate a plurality of polypeptides comprising the polypeptide.

17. The method of claim 16, wherein step of providing the polypeptide further comprises coupling an N-terminal amine group of an N-terminal amino acid (NTAA) residue of the polypeptide to a solid support.

18. A method of analyzing a polypeptide comprising at least one lysine residue, the method comprising the steps of:
a. providing a conjugate of the polypeptide and a recording tag on a solid support, wherein the recording tag comprises a polynucleotide that is conjugated to the polypeptide according to the following steps: (i) contacting the polypeptide with an acylating agent of Formula (I) 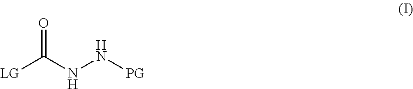
wherein: LG is a leaving group; and PG is a nitrogen protecting group, to attach a semicarbazide group to the at least one lysine residue to form a polypeptide semicarbazide compound; (ii) optionally, removing any protecting group present on the semicarbazide group of the polypeptide semicarbazide compound; and (iii) contacting the polypeptide semicarbazide compound with an ortho-acyl phenylboronic acid of the formula: 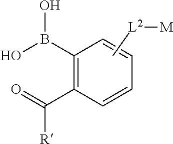
wherein L² is a linking group and M is the recording tag or a reactive functional moiety that is configured to connect the conjugate to the recording tag, under conditions where the semicarbazide group reacts with the ortho-acyl phenylboronic acid to form a diazaborine to provide the conjugate; (iv) optionally, when M is the reactive functional moiety, using the reactive functional moiety to connect the conjugate to the recording tag; (v) attaching the polypeptide or the conjugate to the solid support before or after any one of the steps (i)-(iv);
b. contacting the polypeptide with a binding agent capable of binding to the polypeptide, wherein the binding agent comprises (i) a coding tag that comprises identifying information regarding the binding agent; or (ii) a detectable label; and
c. analyzing the polypeptide by (i) obtaining signal from detectable label upon binding of the binding agent to the polypeptide; or (ii) c1) transferring the identifying information from the coding tag to the recording tag to generate an extended recording tag; and c2) analyzing the extended recording tag.

19. The method of claim 18, wherein the nitrogen protecting group is a carbamate, sulfonamide or acyl.

20. The method of claim 18, wherein analyzing the polypeptide comprises identifying an amino acid sequence of the polypeptide.
